(19) United States
(12) Patent Application Publication
(10) Pub. No.: US 2024/0203594 A1
(43) Pub. Date: Jun. 20, 2024

(54) DECISION-MAKING ASSISTANT TOOL

(71) Applicant: Laboratory Corporation of America Holdings, Burlington, NC (US)

(72) Inventors: Lance Berberian, Burlington, NC (US); Enno de Vries, Tower Lakes, IL (US); Jessie Lunk, Durham, NC (US); Glenn Mogolowitz, Burlington, NC (US)

(21) Appl. No.: 18/542,391

(22) Filed: Dec. 15, 2023

Related U.S. Application Data
(60) Provisional application No. 63/387,859, filed on Dec. 16, 2022.

(51) Int. Cl. G16H 50/20; G06F 16/215; G06F 16/25; G16H 40/20

(52) U.S. Cl. CPC G16H 50/20; G06F 16/215; G06F 16/25; G16H 40/20

(57) ABSTRACT

Techniques for implementing a decision-making assistant tool are disclosed herein. In one particular aspect, identity information about a first entity and contextual information about a second entity are receiving. Subsequently, the decision-making assistant tool can be initialized. A first set of data can be requested from a first computing system by the decision-making assistant tool. A request for a second set of data can be transmitted to a second computing system disjunctive with the first computing system and by the decision-making assistant tool. The first set of data and the second set of data can be de-duplicated to generate de-duplicated data. The de-duplicated data can be provided to the first entity.

### CROSS-REFERENCE TO RELATED APPLICATION

This application claims priority to U.S. Provisional Patent Application No. 63/387,859, filed on Dec. 16, 2022, entitled “DIAGNOSTIC ASSISTANT TOOL” which is hereby incorporated by reference in its entirety.

### FIELD

The present disclosure relates generally to medical records systems, and more particularly, to a computer-based tool for assisting with medical decisions and general medical care.

### BACKGROUND

In the medical field, there is an ever-increasing amount of data. In particular, medical data is collected each time a patient visits a doctor, undergoes a medical procedure or test, and the like. Many regulations exist that inhibit the free flow of data relating to a patient. For example, the patient may separately visit a first doctor, may separately visit an outpatient facility to undergo a medical test, and may separately be admitted to a hospital. In some cases, the first doctor, the outpatient facility, and the hospital may not be configured to share the data about the patient relating to the doctor visit, the outpatient facility, and/or the hospital. Accordingly, it may be difficult for a medical professional to provide quality care to the patient without all available data relating to the patient.

### BRIEF SUMMARY

Techniques disclosed herein relate generally to computer systems. More specifically and without limitation, techniques disclosed herein relate to a decision-making assistant tool, such as a diagnostic assistant tool, that can facilitate decisions of a first entity, such as a medical professional, on behalf of a second entity, such as a patient, using data from disjunctive sources of data. In other examples, techniques disclosed herein relate to a decision-making assistant tool that can facilitate interactions between the first entity and the second entity.

In accordance with aspects of the present disclosure, a system is provided. The system can include a processor and a non-transitory computer-readable medium comprising instructions that are executable by the processor to cause the processor to perform various operations. The system can receive identity information about a first entity and contextual information about a second entity. The system can request a first set of data relating to the second entity from a first computing system using a diagnostic assistant tool. The system can transmit a request for a second set of data relating to the second entity to a second computing system that is disjunctive with respect to the first computing system. The system can receive the first set of data relating to the second entity from the first computing system and the second set of data from the second computing system. The system can de-duplicate the first set of data and the second set of data to generate de-duplicated data about the second entity. The system can transmit the de-duplicated data about the first entity to the diagnostic assistant tool to facilitate an interaction between the second entity and the first entity.

In some embodiments, the first entity includes a patient, and wherein the second entity includes a medical professional configured to provide medical care to the patient.

In some embodiments, wherein the identity information about the first entity comprises personally identifiable information comprising at least one of a name of the first entity, a date of birth of the first entity, an address of the first entity, an email of the first entity, a telephone number of the first entity, a passport number of the first entity, a fingerprint of the first entity, a driver license number of the first entity, a credit card number of the first entity, a debit card number of the first entity, and a Social Security number of the first entity, and wherein the contextual information about the second entity includes at least one of a reason for the first entity visiting the second entity and a type of the second entity.

In some embodiments, the system can additionally (i) receive first input including the identity information about the first entity and the contextual information about the second entity, (ii) receive second input from the second entity that indicates that the second entity selected the diagnostic assistant tool, and (iii) initialize, in response to receiving the second input, the diagnostic assistant tool.

In some embodiments, the system can additionally (i) receive first input including the identity information about the first entity and the contextual information about the second entity, and (ii) automatically initialize, in response to receiving the first input, the diagnostic assistant tool.

In some embodiments, the first computing system includes an electronic medical records system, wherein the second computing system includes a provider database associated with a provider of the diagnostic assistant tool, and wherein the first set of data and the second set of data at least partially overlap.

In some embodiments, the operation of transmitting the de-duplicated data comprises (i) generating, via the diagnostic assistant tool, a user interface, and (ii) providing the de-duplicated data about the first entity via the user interface.

In accordance with aspects of the present disclosure, a method is provided. The method includes receiving, by a computing device, identity information about a first entity and contextual information about a second entity. The method includes requesting, by the computing device, a first set of data relating to the second entity from a first computing system using a diagnostic assistant tool. The method includes transmitting, by the computing device, a request for a second set of data relating to the second entity to a second computing system that is disjunctive with respect to the first computing system. The method includes receiving, by the computing device, the first set of data relating to the second entity from the first computing system and the second set of data from the second computing system. The method includes de-duplicating, by the computing device, the first set of data and the second set of data to generate de-duplicated data about the second entity. The method includes transmitting, by the computing device, the de-duplicated data about the first entity to the diagnostic assistant tool to facilitate an interaction between the second entity and the first entity.

In accordance with aspects of the present disclosure, a non-transitory computer-readable medium comprises instructions that are executable by a processing device for causing the processing device to perform various operations. The operations include receiving identity information about a first entity and contextual information about a second entity. The operations include requesting a first set of data relating to the second entity from a first computing system using a diagnostic assistant tool. The operations include transmitting a request for a second set of data relating to the second entity to a second computing system that is disjunctive with respect to the first computing system. The operations include receiving the first set of data relating to the second entity from the first computing system and the second set of data from the second computing system. The operations include de-duplicating the first set of data and the second set of data to generate de-duplicated data about the second entity. The operations include transmitting the de-duplicated data about the first entity to the diagnostic assistant tool to facilitate an interaction between the second entity and the first entity.

In accordance with aspects of the present disclosure, a method is provided. The method includes receiving, by a diagnostic assistant tool, input indicating a request for resources relating to a first entity, generating, by the diagnostic assistant tool and based on the input indicating the request, a plurality of requests for resources relating to the first entity, transmitting, by the diagnostic assistant tool, each request of the plurality of requests to a corresponding source of disjunctive data of a plurality of sources of disjunctive data, each corresponding source of disjunctive data including a different subset of data relating to the first entity, and receiving, by the diagnostic assistant tool, one or more sets of data relating to the first entity from the plurality of sources of disjunctive data, each set of data of the one or more sets of data originating from a different source of disjunctive data of the plurality of sources of disjunctive data. The method also includes de-duplicating, by the diagnostic assistant tool, the one or more sets of data relating to the first entity to generate a de-duplicated set of data relating to the first entity, transmitting, by the diagnostic assistant tool, the de-duplicated set of data relating to the first entity to an eligibility determination service, receiving, by the diagnostic assistant tool, an eligibility assessment dataset from the eligibility determination service, the eligibility assessment dataset indicating an eligibility of the first entity, and providing, by the diagnostic assistant tool, the eligibility assessment dataset to a second entity for determining the eligibility of the first entity.

In some embodiments, receiving the input indicating a request for resources relating to a first entity includes receiving, by the diagnostic assistant tool, a request for a set of clinical trials for which the first entity is eligible. Each request of the plurality of requests can include a request for an entity lookup for the corresponding source of disjunctive data to execute, and wherein the entity lookup involves identifying data included in the corresponding source of disjunctive data that relates to the first entity. Transmitting the de-duplicated set of data relating to the first entity to the eligibility determination service can include at least partially redacting, by the diagnostic assistant tool, the de-duplicated set of data relating to the first entity to generate a compliant de-duplicated set of data, and wherein the compliant de-duplicated set of data excludes personally identifiable information relating to the first entity.

In some embodiments, the eligibility assessment dataset includes one or more clinical trials and one or more corresponding indications of eligibility, and wherein the one or more corresponding indications of eligibility indicate a likelihood of the first entity being eligible to participate in a clinical trial of the one or more clinical trials corresponding to the likelihood. The one or more sets of data can include at least two sets of data, wherein the at least two sets of data include a first set of data originating from a first source of disjunctive data of the plurality of sources of disjunctive data and a second set of data originating from a second source of disjunctive data of the plurality of sources of disjunctive data, and wherein the first set of data and the second set of data at least partially overlap. De-duplicating the one or more sets of data relating to the first entity can include generating, by the diagnostic assistant tool, a combined set of data by combining the first set of data and the second set of data and de-duplicating, by the diagnostic assistant tool, the combined set of data by causing a mode of each data point included in the combined set of data to be one, wherein, subsequent to being de-duplicating, the combined set of data is the de-duplicated set of data.

In accordance with aspects of the present disclosure, a method is provided. The method includes receiving, by an eligibility determination service, a request from a particular entity for a set of data relating to a set of entities and initiating, by the eligibility determination service, a batch process that involves generating the set of data by transmitting a plurality of requests to a plurality of sources of disjunctive data. The method also includes receiving, by the eligibility determination service, a de-duplicated set of data relating to the set of entities, the de-duplicated set of data generated by a diagnostic assistant tool de-duplicating a plurality of sets of data relating to the set of entities, each set of data of the plurality of sets of data originating from a different source of disjunctive data of the plurality of sources of disjunctive data, generating, by the eligibility determination service and based on the de-duplicated set of data relating to the set of entities, the set of data relating to the set of entities, and providing, by the eligibility determination service, the set of data relating to the set of entities to the particular entity.

In some embodiments, receiving the request from the particular entity for the set of data relating to the set of entities includes receiving, by the eligibility determination service, a request for a set of patients that are eligible to participate in a particular clinical trial. Generating the set of data relating to the set of entities can include determining, by the eligibility determination service, a likelihood that each entity of the set of entities is eligible to participate in the particular clinical trial. Providing the set of data relating to the set of entities to the particular entity can include providing, by the eligibility determination service, the likelihood that each entity of the set of entities is eligible to participate in the particular clinical trial to the particular entity.

In some embodiments, the particular entity includes a medical professional, wherein the set of entities includes a set of patients associated with the medical professional, and wherein providing the set of data relating to the set of patients to the medical professional includes facilitating, by the eligibility determination service, one or more decisions of the medical professional on behalf of the set of patients. The one or more decisions can include, for each patient of the set of patients, determining, based on the set of data, whether each patient is eligible to participate in the particular clinical trial. The batch process can include asynchronously initiating, by the eligibility determination service, the batch process for generating the set of data by asynchronously transmitting the plurality of requests to the plurality of sources of disjunctive data.

In accordance with aspects of the present disclosure, a method is provided. The method includes receiving, by an eligibility determination service, an automatic request from a particular service module for a set of data relating to a set of entities and initiating, by the eligibility determination service, a batch process that involves generating the set of data by transmitting a plurality of requests to a plurality of sources of disjunctive data. The method also includes receiving, by the eligibility determination service, a de-duplicated set of data relating to the set of entities, the de-duplicated set of data generated by a diagnostic assistant tool de-duplicating a plurality of sets of data relating to the set of entities, each set of data of the plurality of sets of data originating from a different source of disjunctive data of the plurality of sources of disjunctive data, generating, by the eligibility determination service and based on the de-duplicated set of data relating to the set of entities, the set of data relating to the set of entities; and providing, by the eligibility determination service, the set of data relating to the set of entities to the particular service module.

In some embodiments, the automatic request from the particular service module for the set of data relating to the set of entities includes receiving, by the eligibility determination service, a request for a set of patients that are eligible to participate in a particular clinical trial. The set of data relating to the set of entities can include determining, by the eligibility determination service, a likelihood that each entity of the set of entities is eligible to participate in the particular clinical trial. The set of data relating to the set of entities to the particular service module can include providing, by the eligibility determination service, the likelihood that each entity of the set of entities is eligible to participate in the particular clinical trial to the particular service module.

In some embodiments, the set of entities includes a set of patients associated with a medical professional, and wherein providing the set of data relating to the set of patients to the particular service module includes facilitating, by the eligibility determination service, one or more decisions of the medical professional on behalf of the set of patients. The one or more decisions can include, for each patient of the set of patients, determining, based on the set of data, whether each patient is eligible to participate in the particular clinical trial. Initiating the batch process can include asynchronously initiating, by the eligibility determination service, the batch process for generating the set of data by asynchronously transmitting the plurality of requests to the plurality of sources of disjunctive data.

In accordance with aspects of the present disclosure, a system is provided. The system can include a processor and a non-transitory computer-readable medium comprising instructions that are executable by the processor to cause the processor to perform various operations. The system can receive identity information about a first entity and contextual information about a second entity. The system can request a first set of data relating to the second entity from a first computing system using a diagnostic assistant tool. The system can transmit a request for a second set of data relating to the second entity to a second computing system that is disjunctive with respect to the first computing system. The system can receive the first set of data relating to the second entity from the first computing system and the second set of data from the second computing system. The system can generate a laboratory report based on the first set of data and the second set of data. The system can generate a graphical user interface (GUI) that augments the laboratory report.

In some embodiments, the first entity includes a patient, and wherein the second entity includes a medical professional configured to provide medical care to the patient.

In some embodiments, the operations can further include automatically populating the GUI with graphical elements based on the first set of data and the second set of data.

In some embodiments, the graphical elements can include cards. The cards can include a therapy considerations card, a clinical trials card, or a discipline specific card.

In some embodiments, the operations can further include prepopulating a response of the questionnaire based on the first set of data and the second set of data.

In some embodiments, the operations can further include prepopulating a response of the questionnaire based on the first set of data and the second set of data.

In some embodiments, the operations can further include receiving a first response of the questionnaire and automatically prepopulating a second response of the questionnaire based on the first response.

In accordance with aspects of the present disclosure, a method is provided. The method includes receiving, by a computing device, identity information about a first entity and contextual information about a second entity. The method includes requesting, by the computing device, a first set of data relating to the second entity from a first computing system using a diagnostic assistant tool. The method includes transmitting, by the computing device, a request for a second set of data relating to the second entity to a second computing system that is disjunctive with respect to the first computing system. The method includes receiving, by the computing device, the first set of data relating to the second entity from the first computing system and the second set of data from the second computing system. The method includes generating, by the computing device, a laboratory report based on the first set of data and the second set of data. The method includes generating, by the computing device, a graphical user interface (GUI) that augments the laboratory report.

In some embodiments, the first entity includes a patient, and wherein the second entity includes a medical professional configured to provide medical care to the patient.

In some embodiments, the method can further include automatically populating, by the computing device, the GUI with graphical elements based on the first set of data and the second set of data.

In some embodiments, the graphical elements can include cards. The cards can include a therapy considerations card, a clinical trials card, or a discipline specific card.

In some embodiments, the method can further include generating, by the computing device, a questionnaire based on the first set of data and the second set of data.

In some embodiments, the method can include prepopulating, by the computing device, at least one response of the questionnaire based on the first set of data and the second set of data.

In some embodiments, the method can further include receiving, by the computing device, a first response of the questionnaire and automatically prepopulating, by the computing device, a second response of the questionnaire based on the first response.

In accordance with aspects of the present disclosure, a system is provided. The system includes one or more processors and a memory coupled to the one or more processors, the memory storing a plurality of instructions executable by the one or more processors, the plurality of instructions comprising instructions that when executed by the one or more processors cause the one or more processors to perform the method.

In accordance with aspects of the present disclosure, a non-transitory computer-readable memory storing a plurality of instructions executable by one or more processors, the plurality of instructions comprising instructions that when executed by the one or more processors cause the one or more processors to perform the method.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 is a block diagram of components of a diagnostic assistant tool according to some embodiments of the present disclosure.

FIG. 2 is a block diagram of a computing environment with which the diagnostic assistant tool of FIG. 1 can be used according to some embodiments of the present disclosure.

FIG. 3 is a block diagram of virtual private clouds that can be used with respect to the diagnostic assistant tool of FIG. 1 according to some embodiments of the present disclosure.

FIG. 4 is a block diagram of security features of the computing environment of FIG. 2 according to some embodiments of the present disclosure.

FIG. 5 is a block diagram of data flow with respect to the diagnostic assistant tool of FIG. 1 according to some embodiments of the present disclosure.

FIG. 6 is a swim-lane diagram of data flow with respect to the diagnostic assistant tool of FIG. 1 according to some embodiments of the present disclosure.

FIG. 7 is a flowchart of a process for aggregating disjunctive medical data using the diagnostic assistant tool of FIG. 1 according to some embodiments of the present disclosure.

FIG. 8 is a block diagram of data flow with respect to a clinical trial context of the diagnostic assistant tool of FIG. 1 according to some embodiments of the present disclosure.

FIG. 9 is a swim-lane diagram of data flow with respect to the clinical trial context of the diagnostic assistant tool of FIG. 1 according to some embodiments of the present disclosure.

FIG. 10 is a flowchart of a process for determining clinical trial applicability and consent using the diagnostic assistant tool of FIG. 1 according to some embodiments of the present disclosure.

FIG. 11 is a block diagram of data flow with respect to a batch-processed clinical trial context of the diagnostic assistant tool of FIG. 1 according to some embodiments of the present disclosure.

FIG. 12 is a swim-lane diagram of data flow with respect to the batch-processed clinical trial context of the diagnostic assistant tool of FIG. 1 according to some embodiments of the present disclosure.

FIG. 13 is a flow chart of a process for determining clinical trial applicability using the diagnostic assistant tool of FIG. 1 according to some embodiments of the present disclosure.

FIG. 14A is an example of a user interface displaying an interactive feature for launching the diagnostic assistant tool of FIG. 1 according to some embodiments of the present disclosure.

FIG. 14B is an example of a user interface displaying the diagnostic assistant tool of FIG. 1 according to some embodiments of the present disclosure.

FIG. 14C is an example of a user interface displaying features of a trend capability of the diagnostic assistant tool of FIG. 1 according to some embodiments of the present disclosure.

FIG. 14D is an example of a user interface displaying additional features of the trend capability of the diagnostic assistant tool of FIG. 1 according to some embodiments of the present disclosure.

FIG. 15A is an example of a user interface displaying a digital dashboard of the diagnostic assistant tool of FIG. 1 according to some embodiments of the present disclosure.

FIG. 15B is an example of a user interface displaying a comments card of the digital dashboard of FIG. 15A according to some embodiments of the present disclosure.

FIG. 15C is an example of a user interface displaying an expanded therapy considerations card of the digital dashboard of FIG. 15A according to some embodiments of the present disclosure.

FIG. 15D is an example of a user interface displaying an expanded clinical trials card of the digital dashboard of FIG. 15A according to some embodiments of the present disclosure.

FIG. 16A is an example of a user interface displaying a clinical decision support (CDS) questionnaire for the diagnostic assistant tool of FIG. 1 according to some embodiments of the present disclosure.

FIG. 16B is an example of a user interface displaying an expanded question card of the CDS questionnaire of FIG. 16A according to some embodiments of the present disclosure.

FIG. 17 is block diagram of a computing device for use with the diagnostic assistant tool in accordance with various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

Certain aspects and features of the present disclosure relate to a diagnostic assistant tool that can be used to aid in providing medical diagnoses and general medical care. The diagnostic assistant tool can be used to aggregate data from disjunctive sources and to convey the aggregated data in a manner that provides assistance to medical professionals in rendering diagnoses and general medical care to a patient or to otherwise facilitate interactions between entities. The diagnostic assistant tool can include one or more software modules, one or more hardware configurations, or a combination thereof. For example, the diagnostic assistant tool can include one or more software modules that can be accessed or initiated from within a user interface. In one such example, a medical provider can interact with a user interface of a medical system that is integrated with the diagnostic assistant tool, such as an electronic health record (EHR) system or an electronic medical record (EMR) system, to launch the diagnostic assistant tool within the user interface. In other examples, the diagnostic assistant tool can be a standalone software module or may otherwise be implemented in hardware.

In some embodiments, the diagnostic assistant tool aggregates medical data from disjunctive sources. The disjunctive sources can include a combination of EHR systems, EMR systems, outpatient facilities, such as testing facilities, and any other suitable disjunctive sources. Other systems, such as the EHR system or EMR system, may not have access to data from each of the disjunctive sources. For example, a doctor may be able to access data relating to a patient from an EHR system or EMR system but may not be able to access data relating to the patient from an outpatient facility or other related testing facilities. Thus, traditionally, to access the data relating to the patient from the outpatient facility or other related testing facilities, the doctor or the patient may submit a records request to the outpatient facility or other related testing facilities to receive the relevant data. However, the records request may take an extended period of time over hours, days, or weeks to be granted. Additionally, during the extended period of time, medical conditions or treatments with respect to the patient may change. Accordingly, without access to the most recent and relevant data relating to the patient from the disjunctive sources, the doctor may not be able to provide appropriate diagnoses or other medical care to the patient.

Additionally, the diagnostic assistant tool can be used in the context of clinical trials. For example, the diagnostic assistant tool can be used to determine eligibility, consent, or a combination thereof for a patient relating to a particular clinical trial. Clinical trials are essential for progress in the medical field, and without clinical trials, life-saving and other beneficial pharmaceuticals, treatments, and the like would not be widely available. Determining eligibility for the patient to participate in the particular clinical trial can be difficult. For example, the particular clinical trial may involve various variables and restrictions that limit the available population eligible to participate in the trial. Without data from the disjunctive sources, the doctor may not be able to make an accurate judgement regarding whether the patient is eligible for the particular clinical trial.

The diagnostic assistant tool can address the above-described difficulties and additional difficulties. For example, the diagnostic assistant tool can retrieve data for a patient from various disjunctive sources. In one such example, the diagnostic assistant tool can communicate with the EMR system or EHR system, the outpatient facility, and any other suitable entity that generates or stores data relating to the patient. The diagnostic assistant tool can request and receive data from the disjunctive sources, can de-duplicate the received data, and can provide assistance to a medical professional in providing a medical diagnosis or otherwise providing general medical care. The diagnostic tool can look for and address non-conformities in the received data. For example, the diagnostic tool can confirm that aspects, such as units, of the received data comply with industry standards for the data. If a portion of the received data does not conform with the rest of the received data, the diagnostic tool can modify the portion of the received data so that the portion is more consistent with the rest of the received data. For example, if the portion of the received data includes units that do not match industry standards for units, the diagnostic tool can perform a unit conversion on the received data. For example, the diagnostic assistant can generate a user interface that displays the received data on trend charts that allow the medical professional to make better-informed decisions compared to other techniques. Additionally, the diagnostic assistant can provide obscure or otherwise difficult-to-find data that can help the medical professional render a more accurate medical diagnosis or more effective medical care compared to other techniques.

The following illustrative examples are presented to introduce the reader to the general subject matter discussed herein and are not intended to limit the scope of the disclosed concepts. The following sections describe various additional features and examples with reference to the drawings in which like numerals indicate like elements and directional descriptions are used to describe the illustrative aspects but, like the illustrative aspects, should not be used to limit the present disclosure. Additionally, the presented figures are generally described with respect to medical techniques and subject matter, but the general subject matter discussed herein is not limited to medical techniques and subject matter.

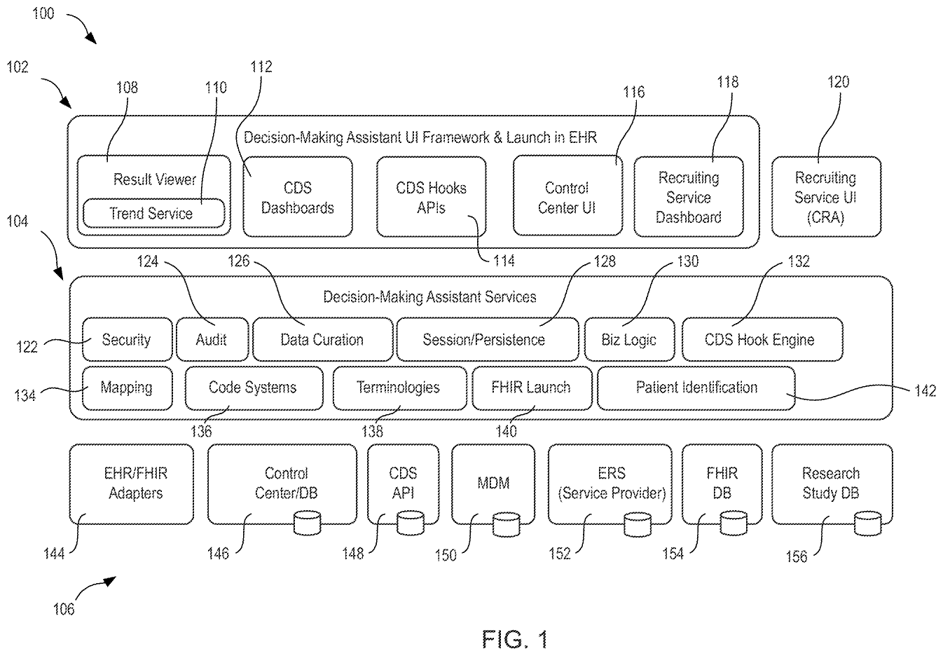

FIG. 1 is a block diagram of components of a diagnostic assistant tool 100 according to some embodiments of the present disclosure. As illustrated, the diagnostic assistant tool 100 includes frontend services 102, backend services 104, and databases 106. The diagnostic assistant tool 100 can include any other suitable components for providing information in a manner to assist a medical professional in rendering a medical diagnosis or general medical care, etc.

In some embodiments, the frontend services 102 of the diagnostic assistant tool 100 includes a user interface. The user interface can receive input data, can output data, and can perform other suitable tasks relating to the diagnostic assistant tool 100. In one such example, the user interface can be used by an entity, such as a medical professional, to access one or more services provided by the diagnostic assistant tool 100. As described herein, the entity is referred to as a medical professional, but this is not intended to be limiting and any user authorized to access the system can do so. In some embodiments, the user interface of the frontend services 102 can provide access to services including a result viewer 108, a trend application 110, a CDS dashboard 112, any other suitable services, or any suitable combination thereof.

The result viewer 108 can include a service of the frontend services 102 that provides results, such as for display or other suitable use, for a patient to the medical professional. For example, the medical professional can input information, such as demographic or identity information, a reason for visiting the medical professional, etc., relating to the patient via the user interface, and the result viewer 108 can provide results, such as medical test results, etc., for the patient. Additionally, the result viewer 108 can include the trend application 110, which may graphically provide results received, for example, via the result viewer 108. The trend application 110 can use the results for the patient to generate a plot of the results over time. The plot can include various types of results corresponding to various types of medical conditions that the medical professional can indicate are of interest.

Additionally, the frontend services 102, provided by or via the user interface, can include a CDS Hooks application programming interface (API) 114, a control center user interface (UI) 116, and a Quick Trial Recruit (QTR) dashboard 118. The CDS Hooks API 114 can include an API gateway that can allow the user interface to request or otherwise receive data relating to the patient. In some embodiments, the CDS Hooks API 114 is configured to make and/or receive API calls via a CDS Hook framework. The control center UI 116 can include a user interface or any other suitable component or service for the control center of the diagnostic assistant tool 100. In some embodiments, the control center UI 116 can be used to access the backend services 104 and/or the databases 106 of the diagnostic assistant tool 100. The QTR dashboard 118 may be or include a dashboard that can interface or otherwise be communicatively coupled to a QTR UI 120, which may be separate from the diagnostic assistant tool 100 or the frontend services 102 thereof. For example, the medical professional can access the QTR UI 120 and can use the QTR UI 120 to access the QTR dashboard 118 for determining eligibility and/or consent for the patient with respect to a particular clinical trial. In some embodiments, the QTR UI 120 and/or the QTR dashboard 118 can receive or otherwise access data about the patient for determining eligibility and/or consent for the patient with respect to the particular clinical trial.

As illustrated, the backend services 104, of the diagnostic assistant tool 100, include a security service 122, an audit service 124, a data curation service 126, a session/persistence service 128, a logic service 130, a CDS Hook engine service 132, a mapping service 134, a code system service 136, a terminology service 138, a Fast Health Interoperability Resources (FHIR) launch service 140, and a patient identification service 142. The backend services 104 can include any other suitable backend services for enabling the functions of the diagnostic assistant tool 100. The security service 122 can generate security tokens, encrypt data, and perform any other suitable security operations for the diagnostic assistant tool 100. For example, when data is transferred, such as within the diagnostic assistant tool 100, to or from the diagnostic assistant tool 100, etc., the security service 122 can determine a level and/or type of security for the data transfer and can apply the determined level and/or type of security. The audit service 124 can provide services for the diagnostic assistant tool relating to potential audits of data within the diagnostic assistant tool 100. For example, the audit service 124 can periodically conduct internal audits, which may be similar to HIPPA audits, to ensure compliance with relevant regulations. The data curation service 126 can provide one or more services for curating data received about the patient for the medical professional. In one such example, the data curation service 126 can filter data received about the patient to identify data about the patient. For example, the medical professional can provide input, such as medical conditions of the patient, medications taken by the patient, test results of the patient, etc., to the diagnostic assistant tool 100, and the input may indicate an interest in viewing the identified data, which can include test results, historical visits, and the like for the patient. The filtering can be done with respect to certain medical conditions or based on other suitable parameters.

The session/persistence service 128 can determine a persistence of a session of the diagnostic assistant tool 100 initiated by the medical professional. For example, the session/persistence service 128 can determine whether to persist a session of the diagnostic assistant tool 100 subsequent to a visit of the patient with the medical professional. The logic service 130 can provide logic services to the diagnostic assistant tool 100 based on the EMR system or EHR system through which the diagnostic assistant tool 100 was launched. The CDS Hook engine service 132 can generate CDS Hooks. For example, in response to the medical professional inputting various data about the patient into the user interface of the diagnostic assistant tool 100, the CDS Hook engine service 132 can generate corresponding CDS Hooks that can be used to request or otherwise receive data based on the input data relating to the patient. The mapping service 134 can map various data within the diagnostic assistant tool 100. For example, the mapping service 134 can map particular test results for the patient to particular medical conditions for providing the mappings to the medical professional. The code system service 136 can provide underlying code services to the diagnostic assistant tool 100. For example, the code system services 136 can test, validate, update, or otherwise manage the code of the diagnostic assistant tool 100. The terminology service 138 can provide relevant terminologies for the diagnostic assistant tool 100. For example, the terminology service 138 can include a real-time-updated dictionary of terms, such as medical terms, relating to services provided by the diagnostic assistant tool 100. The FHIR launch service 140 can provide FHIR services for the diagnostic assistant tool 100. For example, the FHIR launch service 140 can initiate FHIR communications and perform other suitable tasks relating to FHIR. The patient identification service 142 can use data relating to the patient to identify the patient. For example, based on various personally identifiable information (PII), the patient identification service can identify the patient in addition to other data, such as data included in the disjunctive sources, relevant to the patient.

As illustrated in FIG. 1, the databases 106 of the diagnostic assistant tool 100, include EHR/FHIR adapters 144, a control center database 146, a CDS API 148, a medical decision making (MDM) database 150, a provider database 152, a FHIR database 154, and a research study database 156. The databases 106 can include any other suitable databases for the diagnostic assistant tool 100. The EHR/FHIR adapters 144 can include one or more adapters for allowing the diagnostic assistant tool 100 to access or otherwise request data from the EHR system or EMR system via FHIR communications. The control center database 146 can include a database that stores information relating to the control center of the diagnostic assistant tool 100. For example, the control center database 146 can store configuration data, display data, and the like for the control center of the diagnostic assistant tool 100. The CDS API 148 can include a database for storing information relating to the CDS Hook API 114. For example, the CDS API 148 can store data that can allow the CDS Hook API 114 to generate or execute one or more CDS Hooks based on input from the medical professional. The MDM database 150 can include data that can facilitate the medical professional in making medical decisions with respect to the patient. For example, the MDM database 150 can include mappings that are configured to provide suggestions of medical procedures, medical diagnoses, and the like for the patient based on input data such as patient demographic data, patient test results, patient medical conditions, and the like. The provider database 152 can include data gathered and/or stored by the provider of the diagnostic assistant tool 100. For example, the provider database 152 can include a database for a medical testing entity that provides the diagnostic assistant tool 100 such that the test records of patients of the medical testing entity are included in the provider database 152. The FHIR database 154 can include a database for storing information relating to FHIR communications with respect to the diagnostic assistant tool 100. For example, data included in the FHIR database 154 can be used to initiate FHIR communications within and/or external to the diagnostic assistant tool 100. The research study database 156 can include a database for storing information relating to one or more clinical trials. For example, the research study database 156 can include a continuously updated list of active clinical trials in which the patient may be qualified to participate. In some examples, the QTR dashboard 118 and/or the QTR UI 120 can access the research study database 156 to determine whether the patient is eligible for a particular clinical trial, the data of which may also be included in the research study database 156.

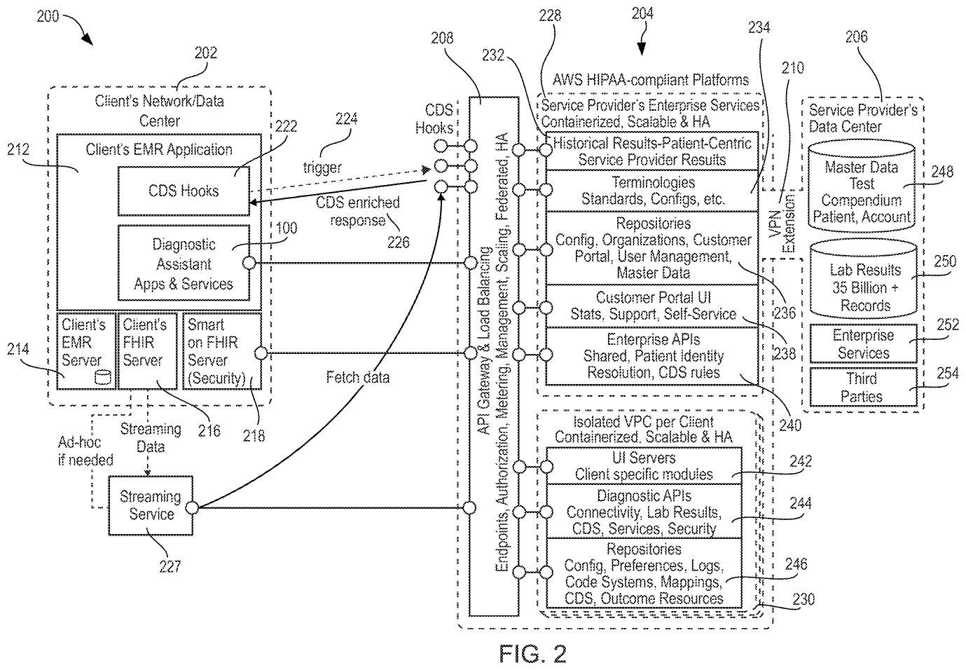

FIG. 2 is a block diagram of a computing environment 200 with which the diagnostic assistant tool 100 can be used according to some embodiments of the present disclosure. As illustrated in FIG. 2, the computing environment 200 includes a client network 202, cloud services 204, a provider network 206, any other suitable components, or any suitable combination thereof. The client network 202 can be communicatively coupled to the cloud services 204, for example, via an API gateway 208, and the cloud services 204 can be communicatively coupled to the provider network 206, which can include a network associated with the provider of the diagnostic assistant tool 100, via a virtual private network (VPN) extension 210.

In some embodiments, the client network 202 includes a client EMR application 212, a client EMR server 214, a client FHIR server 216, and a client security server 218. The client EMR application 212 can include any suitable application that can be used by the medical professional to make or record observations or other suitable data about the patient. Additionally, the client EMR application 212 can execute the diagnostic assistant tool 100. For example, the medical professional can launch the client EMR application 212 upon initiating a visit with the patient and initiate the diagnostic assistant tool 100. The client EMR server 214 can include one or more databases or applications for recording, storing, or performing other suitable tasks with respect to data gathered with respect to the patient. The client FHIR server 216 can provide FHIR communication capabilities for the client network 202, and the client security server 218 can provide security services for FHIR communications initiated within and/or external to the client network 202. The client FHIR server 216 can coordinate with the FHIR database 154 to initiate FHIR communications within and/or external to the diagnostic assistant tool 100

In some embodiments, the client network 202 can communicate with the cloud services 204. For example, via the diagnostic assistant tool 100 or components thereof such as the CDS Hooks API 114 and/or the CDS Hook engine service 132, the client network 202 can generate one or more CDS Hooks 222. The client network 202 can use the CDS Hooks 222 to transmit a trigger 224 to the cloud services 204. The trigger 224 can be transmitted to the API gateway 208, which can allow the cloud services 204 to ingest the trigger 224, and the API gateway 208 can return a CDS enriched response 226 from the cloud services 204 in response to the trigger 224. In some examples, the trigger 224 can include a request for one or more sets of data relating to the patient, and the trigger 224 can be generated or otherwise caused to be generated by the diagnostic assistant tool 100. The CDS enriched response 226 can include the requested one or more sets of data included in the trigger 224. In some embodiments, a streaming service 227 can be used by the client network 202 to continuously communicate with the cloud services 204. For example, the streaming service 227 can access and/or receive data to be continuously streamed to the cloud services 204 via the API gateway 208, and, in response to the client network 202 initiating the trigger 224, the CDS Hooks 222 can retrieve data relevant to the trigger from the streaming service 227.

In some embodiments, the cloud services 204 can include a shared virtual cloud 228 (or more than one virtual cloud), a virtual private cloud 230 (or more than one virtual private cloud), and/or any other suitable types of cloud services. The shared virtual cloud 228 can correspond to a provider environment that can be provided for more than one client of the provider of the diagnostic assistant tool 100. In such examples, the shared virtual cloud 228 can store or otherwise manage data for each of the clients of the provider of the diagnostic assistant tool 100 included in the shared virtual cloud 228. In some embodiments, the data for each of the clients may also be segmented or otherwise not comingled to ensure compliance with various data privacy regulations. As illustrated in FIG. 2, the shared virtual cloud 228 includes historical results 232, terminologies 234, repositories 236, a customer portal UI 238, and enterprise APIs 240, though other suitable components can be included in the shared virtual cloud 228. The historical results 232 can include historical observations, test results, and the like for the patient, and the terminologies 234 can include standards, configurations, and the like for the observations, test results, and the like included in the historical results 232. The repositories 236 can include one or more databases for storing and/or managing data included in the shared virtual cloud 228. For example, the repositories 236 can include one or more repositories, each of which correspond to a different client of the provider of the diagnostic assistant tool 100, etc. The customer portal UI 238 can include a UI for providing statistics, support, and the like for the patient. The enterprise APIs 240 can include patient identity resolutions, CDS rules, and the like for allowing the shared virtual cloud 228 to communicate with separate computing devices, for example via the API gateway 208 and/or the VPN extension 210.

In some embodiments, the virtual private cloud 230 is isolated from the shared virtual cloud 228 and any other services or components included in the cloud services 204. For example, each virtual private cloud 230 included in the cloud services 204 can correspond to a single client of the provider of the diagnostic assistant tool 100 and may not include data from other clients, etc. The isolated nature of the virtual private cloud 230 can allow large clients, such as hospitals, to comply with various data privacy regulations for data that the large clients gather, store, or otherwise manage. As illustrated in FIG. 2, the virtual private cloud 230 includes UI servers 242, diagnostic APIs 244, and repositories 246, though other suitable components can be included in the virtual private cloud 230. The UI servers 242 can include UIs with client-specific modules and/or other suitable components. The diagnostic APIs 244 can include CDS configurations, security considerations, and other suitable components or functionalities for allowing the virtual private cloud 230 to communicate with other suitable computing devices, for example, via the API gateway 208 and/or the VPN extension 210. The repositories 246 can include one or more repositories for the corresponding client of the provider of the diagnostic assistant tool 100, and the one or more repositories can include configuration, preferences, logs, CDS services, security data, and any other suitable information for the virtual private cloud 230. In some embodiments, the virtual private cloud 230 can maintain persistent historical data, for example, to be used for auditing.

The provider network 206 can be communicatively coupled to the cloud services 204 via the VPN extension 210, which can provide secure access to the cloud services 204 for the provider network 206. In some examples, the provider network 206 can communicate with the shared virtual cloud 228 separately with respect to the virtual private cloud 230. Additionally or alternatively, the provider network 206 may indirectly communicate, such as through one or more of the cloud services 204, with the client network 202. As illustrated in FIG. 2, the provider network 206 includes a master database 248, a test results database 250, enterprises services 252, and third-party services 254, though other suitable components and/or services are possible to include with the provider network 206. In some embodiments, the master database 248 includes data relating to the patient and/or other suitable patients that have received services from the provider of the diagnostic assistant tool 100. For example, the data of the master database 248 can include patient identification information, patient account information, and the like. The test results database 250 can include data relating to tests undergone by the patient, and/or other suitable patients, and conducted by the provider of the diagnostic assistant tool 100. The enterprise services 252 and the third-party services 254 can include one or more services provided by the provider of the diagnostic assistant tool 100 and by third parties through the provider of the diagnostic assistant tool 100, respectively.

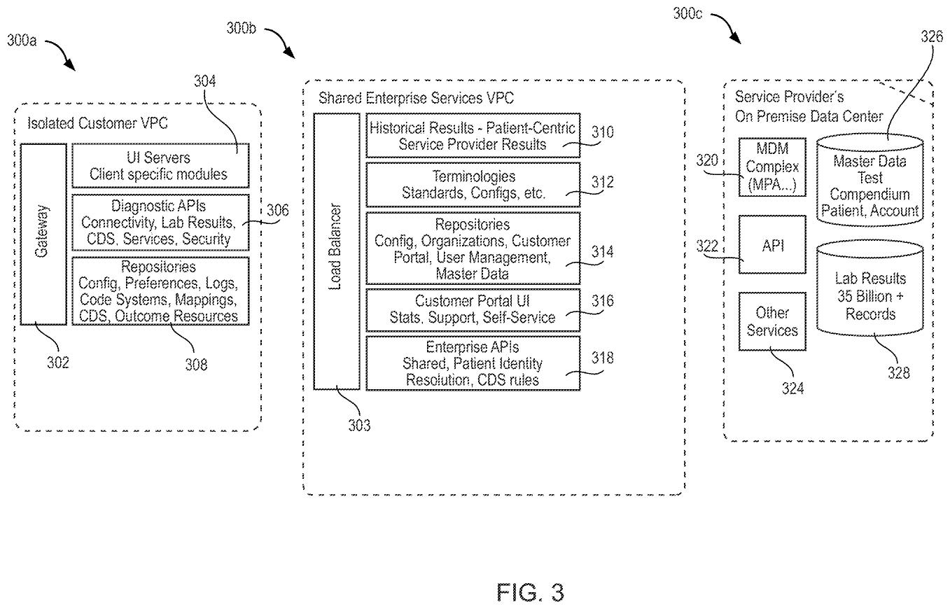

FIG. 3 is a block diagram of isolated systems 300a-c that can be used with respect to the diagnostic assistant tool 100 according to some embodiments of the present disclosure. In some embodiments, the isolated systems can be virtual private clouds. The virtual private clouds can be used to persistently store client data on separate systems such that each client's virtual cloud is not negatively impacted by other clients. For example, if a first client is performing one or more tasks that are computationally expensive, the operation of a second client's tasks may not be negatively impacted since each client's tasks are operated on separate cloud infrastructures. Additionally or alternatively, the data for each of the clients can be saved and accessed separately such that data for different clients are not mixed together as they would be in a shared environment. Although the present disclosure is discussed with respect to the use of isolated systems, such as virtual private clouds, any combination of infrastructures could be used without departing from the scope of the present disclosure.

In some embodiments, the virtual private cloud 300a is similar to identical to the virtual private cloud 230 illustrated and described with respect to FIG. 2, and the virtual private cloud 300b is similar or identical to the shared virtual cloud 228 illustrated and described with respect to FIG. 2. Additionally, the virtual private cloud 300c may be similar or identical to the provider network 206 illustrated and described with respect to FIG. 2.

The virtual private cloud 300a can be configured to support one or more clients of the provider of the diagnostic assistant tool 100. For example, the data included or otherwise exchanges with respect to the virtual private cloud 300a can relate to patients of a single client, and the virtual private cloud 300a may not be configured to receive or otherwise interact with data from other clients of the provider of the diagnostic assistant tool 100. In some embodiments, the virtual private cloud 300a includes a load balancer 302, or alternatively a gateway, UI servers 304, diagnostic APIs 306, and repositories 308, though other suitable components and/or services can be included in the virtual private cloud 300a. The load balancer 302 can allow the virtual private cloud 300a to communicate with separate computing devices. The UI servers 304 may be similar or identical to the UI servers 242 illustrated and described with respect to FIG. 2. For example, the UI servers 304 can include UIs with client-specific modules and/or other suitable components. Additionally or alternatively, the diagnostic APIs 306 can be similar or identical to the diagnostic APIs 244 illustrated and described with respect to FIG. 2. For example, the diagnostic APIs 306 can include CDS configurations, security considerations, and other suitable components or functionalities for allowing the virtual private cloud 300a to communicate with other computing devices. The repositories 308 can be similar or identical to the repositories 246 illustrated and described with respect to FIG. 2. For example, the repositories 308 can include one or more repositories for the client corresponding to the virtual private cloud 300a, and the one or more repositories can include configuration, preferences, logs, CDS services, security data, and any other suitable information for the virtual private cloud 300a.

The virtual private cloud 300b can be configured to support multiple distinct clients of the provider of the diagnostic assistant tool 100. For example, the data included in or otherwise exchanged with respect to the virtual private cloud 300b can relate to patients of the multiple clients, and the virtual private cloud 300b may be configured to receive or otherwise interact with data associated with the multiple clients of the provider of the diagnostic assistant tool 100. In some embodiments, the virtual private cloud 300b includes the load balancer 302, historical results 310, terminologies 312, repositories 314, a customer portal UI 316, and enterprise APIs 318, though other suitable components and/or services can be included in the virtual private cloud 300b. The historical results 310, terminologies 312, repositories 314, a customer portal UI 316, and enterprise APIs 318 may each be similar or identical to the historical results 232, the terminologies 234, the repositories 236, the customer portal UI 238, and the enterprise APIs 240, respectively, illustrated and described with respect to FIG. 2. For example, the historical results 310 can include historical observations, test results, and the like for the patient of the multiple clients, and the terminologies 312 can include standards, configurations, and the like for the observations, test results, and the like included in the historical results 310. Additionally, the repositories 314 can include one or more databases for storing and/or managing data included in the virtual private cloud 300b. For example, the repositories 314 can include one or more repositories, each of which correspond to a different client of the provider of the diagnostic assistant tool 100, etc. The customer portal UI 316 can include a UI for providing statistics, support, and the like for the patients of the multiple clients. The enterprise APIs 318 can include patient identity resolutions, CDS rules, and the like for allowing the virtual private cloud 300b to communicate with separate computing devices.

In some embodiments, the virtual private cloud 300c is similar or identical to the provider network 206 illustrated with respect to FIG. 2. For example, the virtual private cloud 300c can be configured to store data, such as patient identifiable information, test results for the patients, and the like for patients of the provider of the diagnostic assistant tool 100. As illustrated, the virtual private cloud 300c includes an MDM complex 320, various APIs 322, other services 324, a master database 326, and a test results database 328. The MDM complex 320 can be or otherwise include a service for facilitating the medical professional in making decisions about the patient. The various APIs 322 can include one or more APIs that allow the virtual private cloud 300c to communicate with the virtual private clouds 300a-b and/or any other computing device. The other services 324 can include one or more other services suitable for allowing the provider of the diagnostic assistant tool 100, for example, through the virtual private cloud 300c, to provide the functionalities described herein to the multiple clients. In some embodiments, the master database 326 and the test results database 328 are similar or identical to the master database 248 and the test results database 250, respectively, described and illustrated with respect to FIG. 2. For example, the master database 326 can include data relating to the patient and/or other suitable patients that have received services from the provider of the diagnostic assistant tool 100. Additionally, the data of the master database 326 can include patient identification information, patient account information, and the like. The test results database 328 can include data relating to tests undergone by the patient, and/or other suitable patients, and conducted by the provider of the diagnostic assistant tool 100.

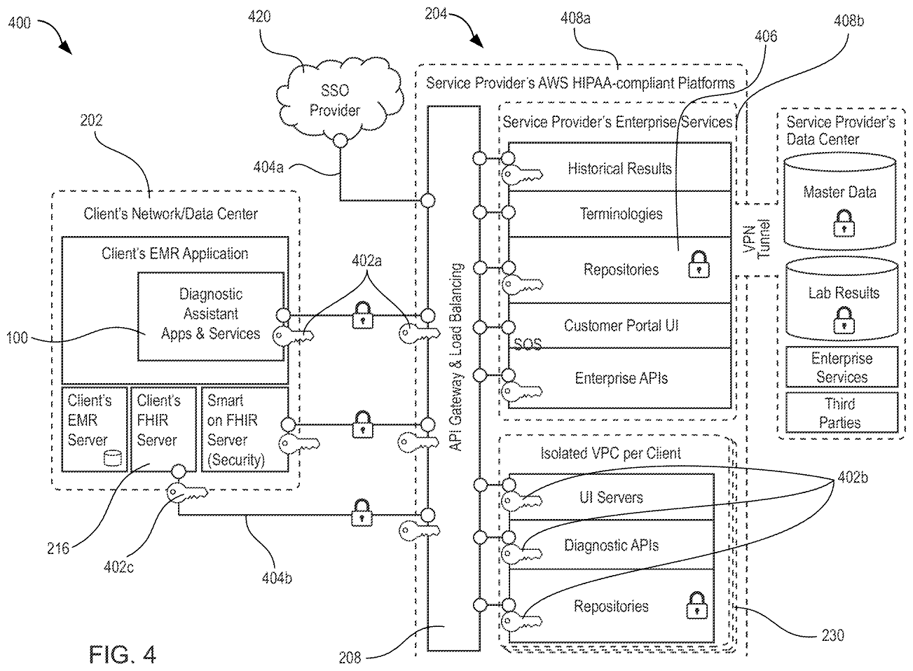

FIG. 4 is a block diagram of security features of the computing environment 200, according to some embodiments of the present disclosure. FIG. 4 illustrates various security features of the computing environment 200. For example, the security features include security tokens, such as tokens 402a-c) service-to-service credentialing, such as encryptions 404a-b, data encryption, such as encryption 406, security zones, such as security zones 408a-b, and other security features. Some of the general security features used by the computing environment 200 may include JSON web tokens (JWTs), secure sockets layer (SSL) encryption, application programming interface (API) authentication, firewalls, in-flight encryption, disc encryption, table encryption, and the like.

In some embodiments, the computing environment 200 uses security tokens to increase security of data exchanges within the computing environment 200. For example, the diagnostic assistant tool 100 can generate and/or receive tokens 402a when communicating with the cloud services 204 via the API gateway 208. Additionally, the virtual private cloud 230 can generate a token 402b when attempting to communicate with the client network 202, and the token 402b can be compared, for example by the client network 202, etc., to the token 402c, which can be generated by the client network 202 to control access to the client network 202, to determine whether any security breaches are indicated by the communication attempt. Additionally or alternatively, the computing environment 200 can use service-to-service credentialing when different components are communicating with one another. For example, the encryption 404a between an SSO provider 420 and the API gateway 208 and the encryption 404b between the client FHIR server 216 and the API gateway 208 can each involve service-to-service credentialing and/or any other encryption techniques. In some embodiments, one or more components of the cloud services 204 and/or the provider network 206 can be encrypted. For example, the encryption 406 can cause the data included in the repositories 236 of the shared virtual cloud 228 to be encrypted. Additionally or alternatively, the computing environment can include security zones 408a-b that involve firewalls or other security techniques within the zones. For example, the security zone 408a, corresponding to the cloud services 204, and the security zone 408b, corresponding to the shared virtual cloud 228, can each include a firewall or other suitable security features surrounding the respective security zone. Other suitable security zones and security features are possible to include in the computing environment 200.

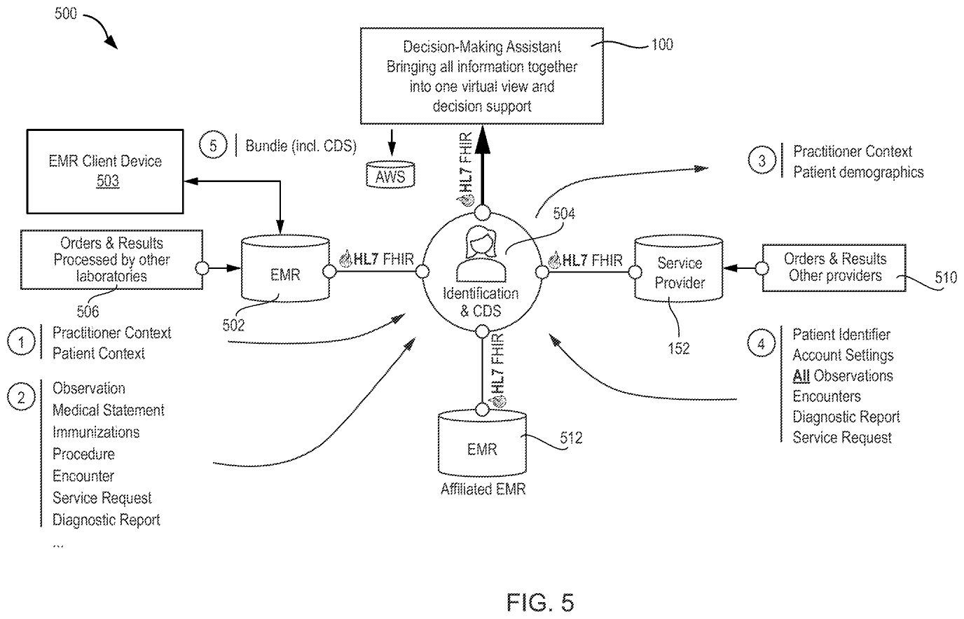

FIG. 5 is a block diagram of data flow with respect to the diagnostic assistant tool 100 of FIG. 1, according to some embodiments of the present disclosure. The data flow diagram 500 can represent a flow of data with respect to a medical professional initiating a medical visit with a patient. In some embodiments, the data flow diagram 500 can be initiated in response to a user, such as a medical professional, using a client device 503 to access the EMR system 502 and/or accessing patient information within the EMR system 502. For example, in response to the medical professional launching user interface for accessing an EMR system 502, on the client device 503, data may flow as illustrated with respect to the data flow diagram 500. In another example, the steps within the data flow diagram 500 can be initiated in response to the medical professional searching for or accessing a patient file within the EMR system 502. In other embodiments, data flow through the data flow diagram 500 can be otherwise suitably initiated. The EMR system 502, for example as discussed herein, can include any combination of electronic medical record system including the application for running and accessing the EMR system as well as the database storing information, such as patient information, within the EMR system.

The data exchange and flow through the data flow diagram 500 may be carried out using a combination of CDS hooks, REST, FHIR protocol(s), and the like. For example, the flow 500 can include a user interface on the client device 503 initiates CDS trigger and/or a REST call to call one or more applications within the diagnostic assistant services 504 to query information, such as patient information, to be cached. In some embodiments, the diagnostic assistant tool 100 can be loaded within a user interface on the client device 530 accessing the EMR system 502, such as within an iframe, for example, the medical professional can initiate a visit with the patient and can login to the EMR system on the client device 503, which can cause the diagnostic assistant tool 100 to be launched therein. This can allow a third-party application to run within the EMR system 502 so the user does not have to leave the EMR applications to view the data from an application such as the diagnostic assistant tool 100. In other examples, the user interface includes an interactive element, such as a button, that, when interacted with, launches the diagnostic assistant tool 100.

In some embodiments, when launching the diagnostic assistant tool 100, a combination of steps can be initiated. As illustrated in FIG. 5, the steps can include: Step 1, providing practitioner context and patient demographics to the diagnostic assistant services 504, for example as part of a service request. The practitioner context and patient demographics can include any combination of information that provides sufficient information to find data related to the practitioner and/or and patient, for example, personal identifier information. The practitioner context and patient demographics can be provided in any combination of methods, for example, as JSON objects that can be returned as FHIR results. At Step 2, subsequent to or substantially contemporaneous with respect to Step 1, the diagnostic assistant services 504 can gather data in background from a combination of local and third-party databases such as affiliated EMRs that have provided permission to use their data to further enrich data. At Step 3, the diagnostic assistant services 504 can use the received practitioner context and patient to identify the patient within the provider database 152. At Step 4, the diagnostic assistant services 504 can receive the test results for an identified patient from the provider database 152. In some embodiments, the test results can include all data for the identified patient, and the data may not necessarily be limited to the information associated with the requesting practitioner. At Step 5, data aggregation can be performed by the diagnostic assistant services 504. In some embodiments, the aggregated data can be stored in a memory cache and can be de-duplicated in real-time such that the aggregated data is not being persistently stored. In other words, the cached data can be aggregated specifically for the received practitioner context and patient context for this transaction such that a similar call for the same practitioner context and patient context may be aggregated again. This provides a technique in which aggregated data can constantly be up-to-date and maintained in real-time. Thereafter, the de-duplicated data can be streamed to EMR 502 in real-time as the data is aggregated and becomes available. These steps can be performed as the medical professional accesses a patient file.

In some embodiments, the EMR system 502 can transmit patient demographic information (patient context), entity context information (practitioner context), or a combination thereof to diagnostic assistant services 504, which may be included in or otherwise executed by the diagnostic assistant tool 100. The patient demographic information can include identifying information for a patient or medical professional. For example, the demographic information can include any combination of information related to a patient's name, date of birth, address, email, telephone number, passport number, fingerprint, driver license number, credit card number, debit card number, Social Security number, etc. Additionally, the demographic information can include height, weight, race, gender, employment history, etc. The entity context information can include contextual information for a provider, medical professional, facility, etc. For example, the context information can include a combination of information related to a reason for visit, type of medical professional, previous visits, etc.

Substantially contemporaneous to launching the EMR system 502 and transmitting patient demographic and entity context information to the diagnostic assistant services 504, the diagnostic assistant services 504 can access the EMR system 502. For example, the diagnostic assistant services 504 can make a call, such as a FHIR-protocol call, a CDS Hook, an API call, etc., to the EMR system 502 for receiving data about the patient from the EMR system 502. The data may include observations, medical statements, immunizations, procedures, encounters, service requests diagnostic reports, etc. for the patient. In some embodiments, the EMR system 502 includes or can otherwise access orders and results 506 relating to the patient. For example, the EMR system 502 can store historical data that includes the previously placed orders and results 506 relating to the patient. In some embodiments, the orders and results 506 includes historical medical tests, historical prescriptions, historical medical procedures, a medical history, and the like relating to the patient. The EMR system 502 can transmit the orders and results 506 relating to the patient to the diagnostic assistant services 504 substantially contemporaneous to transmitting the patient demographic and entity context information to the diagnostic assistant services 504.

The diagnostic assistant services 504 can additionally communicate, such as via FHIR query protocol, etc., with a provider database 152. The provider database 152 can include a database of patient records generated and/or stored by the provider that owns, manages, or otherwise provides the diagnostic assistant tool 100 for the medical professional. The provider database 152 can store orders and records 510 for the patient. In some embodiments, the orders and records 510 included in the provider database 152 may be different than the orders and results 506 included in the EMR system 502. For example, the set of data included in the orders and records 510 may include services or tests not offered by the EMR system owner, were substituted out by the EMR system owner, and/or were performed without knowledge of the EMR system owner, which may be or include a previous doctor, an out of state medical facility, etc. Additionally or alternatively, different providers may store the same data different with different levels of detail. Therefore, while some overlap between data may occur, the data in the orders and records 510 may not be identical to the set of data included in the orders and results 506. Accordingly, the EMR system 502 and the provider database 152 may be disjunctive sources of data for the patient.

The diagnostic assistant services 504 can transmit the patient demographic and entity context information to the provider database 152. In some embodiments, the provider database 152 can perform a search of the orders and records 510 to identify data included in the provider database 152 that relates to the patient. The search at the provider database 152 can be initiated substantially contemporaneous to, or subsequent to, accessing a patient file, such that once the user requests access to the information, it may be populated and/or being populated with the retrieved data. In some embodiments, the provider database 152 identifies the data relating to the patient. The data can include patient account information, patient identifying information, test records, observations, and other suitable data relating to the patient. The provider database 152 can output the identified data relating to the patient and can transmit the data to the diagnostic assistant services 504.

Additionally or alternatively, the diagnostic assistant services 504 can communicate, such as via FHIR query protocol, etc., with other EMR systems 512. The other EMR systems 512 may include one or more EMR systems that may not be affiliated with the EMR system 502 or the provider database 152. For example, the other EMR systems 512 can include parties that are affiliated or otherwise cooperate in sharing data with one of the EMR system 502 or the provider database 152. In some embodiments, the diagnostic assistant services 504 communicates with other disjunctive sources of data that include the EMR systems 512 to query, such as with the patient demographic and entity context information, and/or receive additional data relating to the patient. Accordingly, the diagnostic assistant services 504 can query, for example using the patient demographic and entity context information, the EMR system 502, the provider database 152, and the other suitable disjunctive sources of data, such as the EMR systems 512, and receive disjunctive data relating to the patient.

Additionally or alternatively, the diagnostic assistant services 504 may de-duplicate the received data. In one such example, a first set of data from the EMR system 502 may overlap a second set of data relating to the patient from the provider database 152 and/or a third set of data relating to the patient from any combination of the EMR systems 512. The diagnostic assistant services 504 can generate and maintain a memory cache that aggregates records per patient and de-duplicates the per-patient records. For example, the diagnostic assistant services 504 can use a caching service as a temporary data repository to gather information from many different data sources and processes the information through a deduplication process. The diagnostic assistant services 504 can de-duplicate the received data such that each identical data point that may be included in one or more of the first set of data, the second set of data, and/or the third set of data is ingested once into the diagnostic assistant tool 100. In some embodiments, the memory cache is refreshed (e.g., deleted and regenerated) each time the diagnostic assistant tool 100 is launched for a different patient. In some examples, the diagnostic assistant services 504 can look for and address non-conformities in the received data. For example, the diagnostic tool can confirm that aspects, such as units, of the received data comply with industry standards for the data. If a portion of the received data does not conform with the rest of the received data, the diagnostic tool can modify the portion of the received data so that the portion is more consistent with the rest of the received data. For example, if the portion of the received data includes units that do not match industry standards for units, the diagnostic tool can perform a unit conversion on the received data.

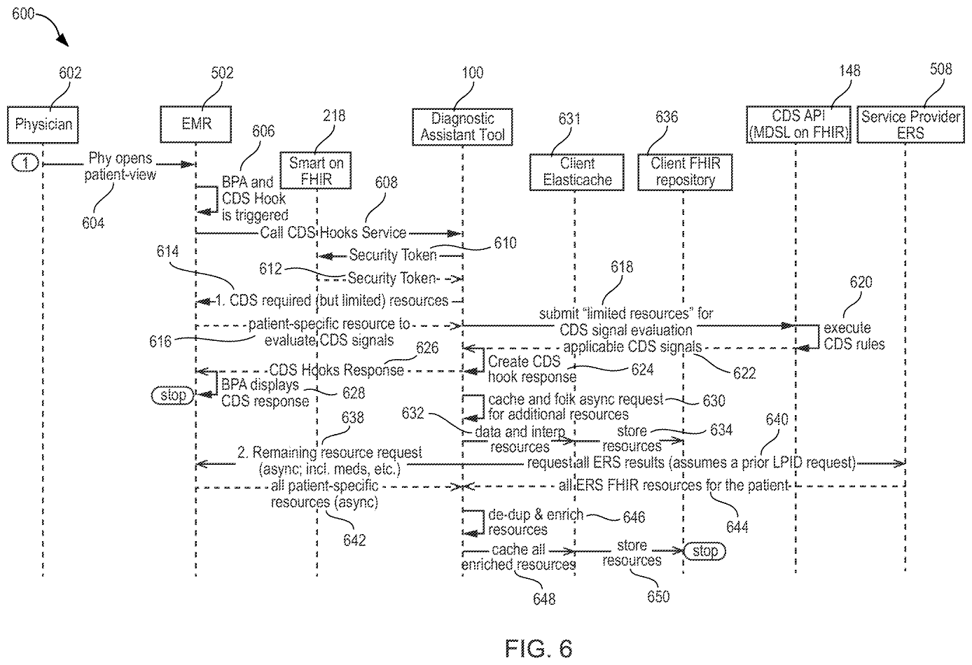

FIG. 6 is a swim-lane diagram 600 of data flow with respect to the diagnostic assistant tool 100, according to some embodiments of the present disclosure. While described in a particular order, the actions and operations described with respect to the swim-lane diagram 600 can be performed in any other suitable order including at least partially substantially contemporaneously. In some embodiments, the swim-lane diagram 600 begins with a medical professional 602 opening a patient view of the EMR system 502 (action 604) for a patient. For example, the patient can visit the medical professional 602, and the medical professional 602 can login to the EMR system 502 using credentials of the medical professional 602. By logging into the EMR system 502, the EMR system 502 can generate a CDS Hook (action 606) and the EMR system 502 can use the CDS Hook to submit a CDS Hook service call (action 608) to the diagnostic assistant tool 100. In response to receiving the CDS Hook service call, the diagnostic assistant tool 100 generates a security token (action 610) and transmits the security token to the client security server 218. The client security server 218 generates a subsequent security token and transmits the subsequent security token to the diagnostic assistant tool 100 in response to receiving the security token from the diagnostic assistant tool 100 (action 612). The diagnostic assistant tool 100 can compare the security token and the subsequent security token. In some embodiments, in which the comparison by the diagnostic assistant tool 100 indicates that the security tokens match or otherwise correspond to one another, the diagnostic assistant tool 100 generates and/or identifies CDS limited resources (e.g., that were requested by the EMR system 502) and transmits the resources to the EMR system 502 (action 614).

The EMR system 502 can transmit patient-specific data or resources to the diagnostic assistant tool 100 (action 616) for evaluating CDS signals. Additionally, the diagnostic assistant tool 100 submits the CDS limited resources, such as described with respect to the action 614, to the CDS API 148 (action 618). The CDS API 148, or any other suitable component of the diagnostic assistant tool 100, can execute CDS rules included in the CDS API 148 (action 620). The CDS rules can include statement assessments with Boolean outcome, which may evaluate conditions around a patient. These conditions could include ranges of measurements, positive or negative outcome, medications, procedures, diagnosis, the delta between two or many encounters over time, the rate of change, and many other clinical, social demographic, or temporal considerations. In other words, the CDS rules can be provided to trigger when deeper analysis may be required, for example, as part of a clinical decision support program. Additionally, the CDS API 148 can transmit applicable CDS signals, which may be based on the CDS rules, to the diagnostic assistant tool 100 (action 622), which can then generate a CDS Hook response (action 624). The diagnostic assistant tool 100 can transmit the CDS Hook response to the EMR system 502 (action 626), and the EMR system 502 can output, such as for display and viewing by the medical professional 602, the CDS Hook response (action 628). In some embodiments, the CDS Hook response can include information relating to the patient that was requested by the medical professional 602.

Additionally, the diagnostic assistant tool 100 can initiate a background or substantially contemporaneous process with respect to the CDS Hook service request (action 608). For example, the diagnostic assistant tool 100 submits an asynchronous request for additional resources relating to the patient (action 630). The diagnostic assistant tool 100 submits resources including patient demographic and entity context information to a client elasticache 631 (action 632), and the client elasticache 631 stores the resources in a client FHIR repository 636 (action 634). In some embodiments, the client elasticache 631 can be part of the customer's virtual cloud network. The client FHIR repository 636 can be the same as or similar to the FHIR DB 154 as illustrated and discussed with respect to FIG. 1. Additionally or alternatively, the diagnostic assistant tool 100 can submit a request to the EMR system 502, such as via EHR/FHIR adapters 144, for the remaining resources, which may be resources not presently stored by the diagnostic assistant tool 100, relating to the patient stored in the EMR system 502 (action 638), and the diagnostic assistant tool 100 submits a request to the provider database 152 for resources relating to the patient stored in the provider database 152 (action 640).

The EMR system 502 identifies the remaining resources stored in the EMR system 502 and requested by the diagnostic assistant tool 100 and transmits the remaining resources to the diagnostic assistant tool 100 (action 642). The provider database 152 identifies the remaining resources stored in the provider database 152 and requested by the diagnostic assistant tool 100 and transmits the remaining resources to the diagnostic assistant tool 100 (action 644). In response to receiving the requested resources from the EMR system 502 and the provider database 152, the diagnostic assistant tool 100 de-duplicates the received resources such that each distinct data point among the requested and received resources exists once in the data provided by the diagnostic assistant tool 100 for the medical professional 602 (action 646). The de-duplicated resources are cached, for example in the client elasticache 631 (action 648), and the cached resources are stored in the client FHIR repository 636 (action 650). In some embodiments, the client elasticache 631 can be constantly and/or periodically refreshed as incoming data is received within the system 100.

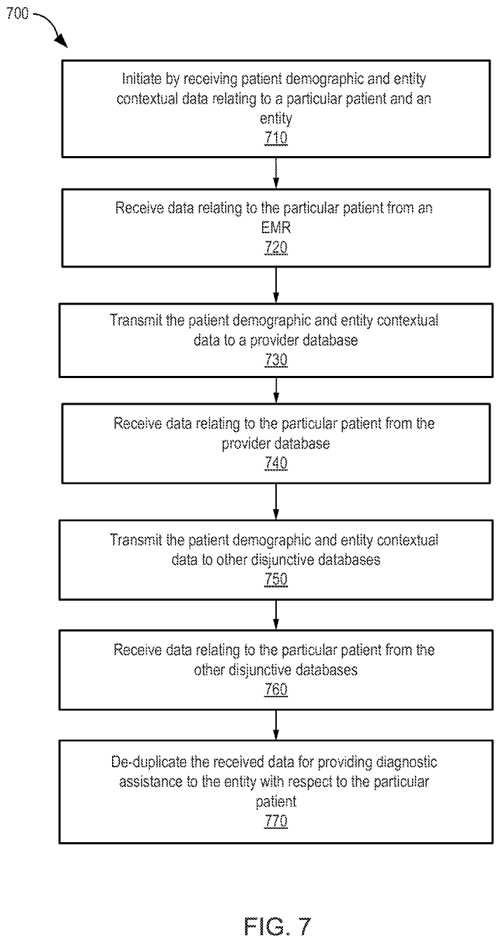

FIG. 7 is a flowchart of a process 700 for aggregating disjunctive medical data using the diagnostic assistant tool 100, according to some embodiments of the present disclosure. At block 710, the diagnostic assistant tool 100 is initiated by receiving patient demographic and entity contextual information. For example, the diagnostic assistant tool 100 can be initiated when a medical professional logs into an EMR system and enters patient demographic information and/or entity contextual information. By logging into the EMR system, demographic information or identity information, such as name, date of birth, and other identifying information, about the patient and contextual information, such as a reason for visit, a type of entity, etc., about the medical professional can be transmitted to the diagnostic assistant tool 100. Accordingly, the diagnostic assistant tool 100 can launch for providing diagnostic assistance to the medical professional with respect to the patient.

At block 720, the diagnostic assistant tool 100 receives data relating to the patient from the EMR system. For example, upon launching, the diagnostic assistant tool 100 can submit a request, such as a FHIR query protocol request, CDS Hook request, etc., to the EMR system for data relating to the patient. The requested data can include historical visits to the medical professional or other visits to other medical professional for which data is available in the EMR system, test records relating to the patient, and other suitable data relating to the patient and stored in the EMR system. In response to receiving the request, the EMR system can identify the requested data and can transmit the requested data to the diagnostic assistant tool 100.

At block 730, the diagnostic assistant tool 100 transmits patient demographic and entity contextual data to a provider database such as a database owned or operated by a service provider and/or a provider of the diagnostic assistant tool 100. In some embodiments, the diagnostic assistant tool 100 transmits the patient demographic and entity contextual data to the provider database using the FHIR query protocol or other suitable data exchange protocol. The provider database can receive the patient demographic and entity contextual data, and the provider database can use the patient demographic and entity contextual data to identify data relating to the patient desired or otherwise requested by the medical professional.

At block 740, the diagnostic assistant tool 100 receives data relating to the patient from the service provider database. The service provider database can identify the data relating to the patient, and the data can include test records, visits to medical professionals, and/or other suitable observations made or otherwise stored by the provider of the diagnostic assistant tool 100 with respect to the patient. In some examples, the service provider database can identify the data relating to the patient by querying patient records included in the service provider database using the patient demographic information and entity context information. In some embodiments, in response to identifying the data relating to the patient desired or otherwise requested by the medical professional, the provider database transmits the data relating to the patient to the diagnostic assistant tool 100. For example, the provider database can transmit the data relating to the patient using the FHIR query protocol or other, suitable and/or similar data exchange protocol.

At block 750, the diagnostic assistant tool 100 transmits patient demographic and entity contextual data to one or more disjunctive sources. The diagnostic assistant tool 100 can identify the one or more disjunctive data sources. For example, the diagnostic assistant tool 100 can identify one or more disjunctive EMR systems, one or more disjunctive medical providers, and/or other suitable sources of disjunctive data relating to the patient. In some embodiments, the diagnostic assistant tool 100 transmits the patient demographic and/or entity contextual data to each of the identified disjunctive data sources using the FHIR query protocol or other suitable data exchange protocol. The identified disjunctive data sources can receive the patient demographic and entity contextual data, and the identified disjunctive data sets can use the patient demographic and entity contextual data to identify data relating to the patient desired or otherwise requested by the medical professional and included in the identified disjunctive data sets. The disjunctive data sets can come from any combination of separate databases.

At block 760, the diagnostic assistant tool 100 receives data relating to the patient from the one or more disjunctive data sources. In some embodiments, the identified disjunctive data sources can identify the data relating to the patient, desired or otherwise requested by the medical professional, and transmit the data relating to the patient to the diagnostic assistant tool 100. For example, the identified disjunctive data sources can transmit the data relating to the patient using the FHIR query protocol or other, suitable and/or similar data exchange protocol. The data received by the diagnostic assistant tool 100 can include test records, visits to medical professionals, and/or other suitable observations made or otherwise stored by one or more entities of the identified disjunctive data sources with respect to the patient.

At block 770, the diagnostic assistant tool 100 de-duplicates the received data for providing information to the medical professional with respect to the patient. In response to receiving the requested data from the EMR system, the provider database, and/or the identified disjunctive data sources, the diagnostic assistant tool 100 de-duplicates the received data such that a mode of each distinct piece of data relating to the patient is a single copy regardless of how many times the distinct piece of data exists in the received data. Accordingly, the diagnostic assistant tool 100 can provide the de-duplicated data relating to the patient to the medical professional for providing assistance to the medical professional for providing diagnoses or general medical care with respect to the patient.

In some embodiments, the de-duplication process can include storing, standardizing, and clensing steps of data collected from multiple sources. The above-described steps can include any combination of identifying record sources, a record provenance, record ordering physicians, record sample, record date and timestamp, and record value, etc. The steps can be used to ensure that separate records, which may involve the same event, such as a test result, are not provided to the user as duplicates. For example, when a test is ordered by the medical professional within the EMR to a service provider, the results can be formatted and stored according to the respective format of the receiving database. Additionally or alternatively, the service provider can save the test results in the provider database using a proprietary identifier or formatting and can transmit the results to the ordering medical professional. The results may then be stored in a local EMR using a respective, proprietary identifier or formatting. While the two test results may not be duplicates, the two tests represent the same test such that they should be deduplicated. In some embodiments, duplicate data sets may be retained and may be identified as duplicates of one antoher and associated with a primary record. Duplicates can be flagged and standardized. For example, data identified as duplicates can be saved as children to a selected parent result such that the parent result is displayed to the user, for example within the diagnostic assistant tool 100 user interface. Using the deduplicaiton process ensures that the result is from a different physical sample (tube, tissue, etc.) and collected not at the same time to distinguish from multiple records systems not synchronizing the data sources.

In some embodiments, the diagnostic assistant tool 100 provides the de-duplicated data to a client device, such as the EMR 502 client, via a stream from a cached memory system such as the client elasticache 631. For example, upon being launched, the diagnostic assistant tool 100 can generate the cached memory system, and the diagnostic assistant tool 100 can cache or otherwise store the received, de-duplicated data from the EMR system, the provider database, and/or the disjunctive data sources in the cached memory system. Subsequently, the diagnostic assistant tool 100 provides the de-duplicated data stored in the cached memory system upon receipt, such as asynchronously and/or substantially contemporaneously, to the EMR client 503. The data is provided to the EMR client 503 such that none of the aggregated data is stored or accessible by the EMR client 503, for viewing through a user interface, for example, as illustrated in FIGS. 4A-4D. Additionally or alternatively, the cached memory system can be refreshed for each initiation, such as for each subsequent patient profile pulled up by a practitioner, of the diagnostic assistant tool 100.

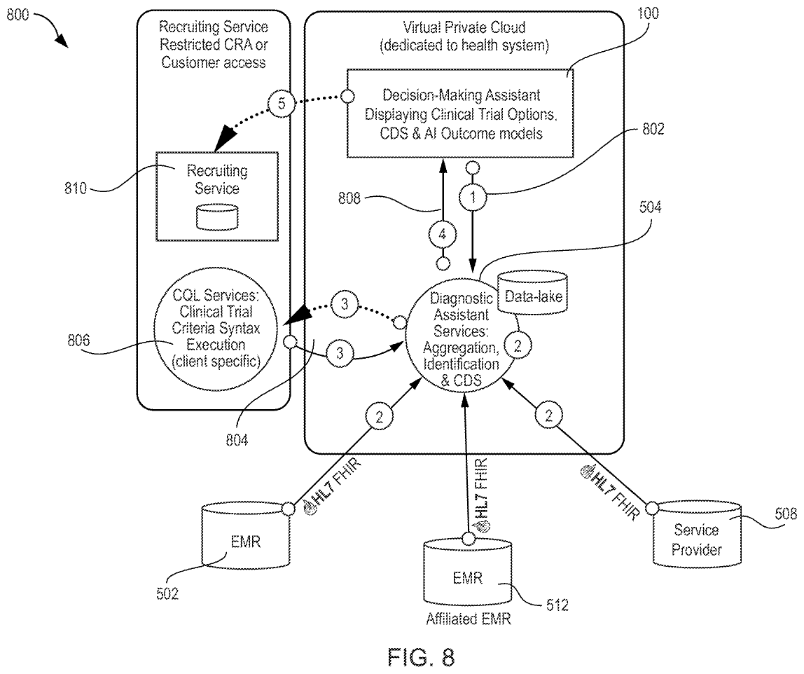

FIG. 8 is a block diagram of data flow with respect to a clinical trial context of the diagnostic assistant tool 100 according to some embodiments of the present disclosure. The data flow diagram 800 can represent a flow of data with respect to a medical professional initiating a CDS Hook relating to clinical trials for a patient. For example, in response to the medical professional launching the diagnostic assistant tool 100, the medical professional can use the diagnostic assistant tool 100 to initiate the CDS Hook, which may cause data to flow as illustrated with respect to the data flow diagram 800. In other embodiments, data flow through the data flow diagram 800 can be otherwise suitably initiated including automatically upon initiating the diagnostic assistant tool 100. Additionally or alternatively, data exchange and flow through the data flow diagram 800 may be carried out using FHIR query and/or any other suitable protocol(s).

In some embodiments, a first data exchange 802 is completed between the diagnostic assistant tool 100 and the diagnostic assistant services 504. For example, the first data exchange 802 can involve a CDS Hook trigger being generated by the diagnostic assistant tool 100 and transmitted to the diagnostic assistant services 504 for providing identification of the patient and identification of clinical trials to the diagnostic assistant services 504. Subsequently, the diagnostic assistant services 504 can perform patient matching techniques. For example, the diagnostic assistant services 504 can compare patient identification information from the CDS Hook trigger to available data. Additionally or alternatively, the diagnostic assistant services 504 submits a request, for example to the EMR system 502, the provider database 152, and/or the disjunctive data sources 512, a lookup request, which may cause the EMR system 502, the provider database 152, and/or the disjunctive data sources 512 to identify and transmit respective data relevant to the patient and/or various clinical trials to the diagnostic assistant services 504.

In some embodiments, the results of the lookup request are used by the diagnostic assistant services 504 to determine an eligibility of the patient participating in one or more clinical trials. For example, the diagnostic assistant services 504 can perform a second data exchange 804 by transmitting the lookup results, relating to the patient, to clinical quality language (CQL) services 806. The CQL services 806 can compare the results from the patient lookup against a set of clinical trials. In some embodiments, the results from the patient lookup include a medical history such as medical conditions of the patient, medical tests of the patient, and the like. Accordingly, the CQL services 806 can compare the results from the patient lookup against various eligibility parameters of the clinical trials, and the CQL services 806 can determine a set of clinical trials for which the patient is eligible. In some embodiments, the set of clinical trials includes zero clinical trials, one clinical trial, two clinical trials, or any other suitable number of clinical trials. The CQL services 806 can transmit the set of clinical trials to the diagnostic assistant services 504.

In response to receiving the set of clinical trials, the diagnostic assistant services 504 can perform a third data exchange 808 in which the diagnostic assistant services 504 transmits the set of clinical trials to the diagnostic assistant tool 100. The diagnostic assistant tool 100 provides the set of clinical trials to the medical professional for facilitating a decision by the medical professional for whether the patient can participate in one or more clinical trials included in the set of clinical trials. In some embodiments, the medical professional receives consent from the patient for participating in a clinical trial of the set of clinical trials. Accordingly, the medical professional can input the consent of the patient relating to the clinical trial into the diagnostic assistant tool 100, and the diagnostic assistant tool 100 transmits the consent of the patient with respect to the clinical trial to a QTR database 810 that is configured to store and/or manage consent of patients with respect to participating in respective clinical trials.

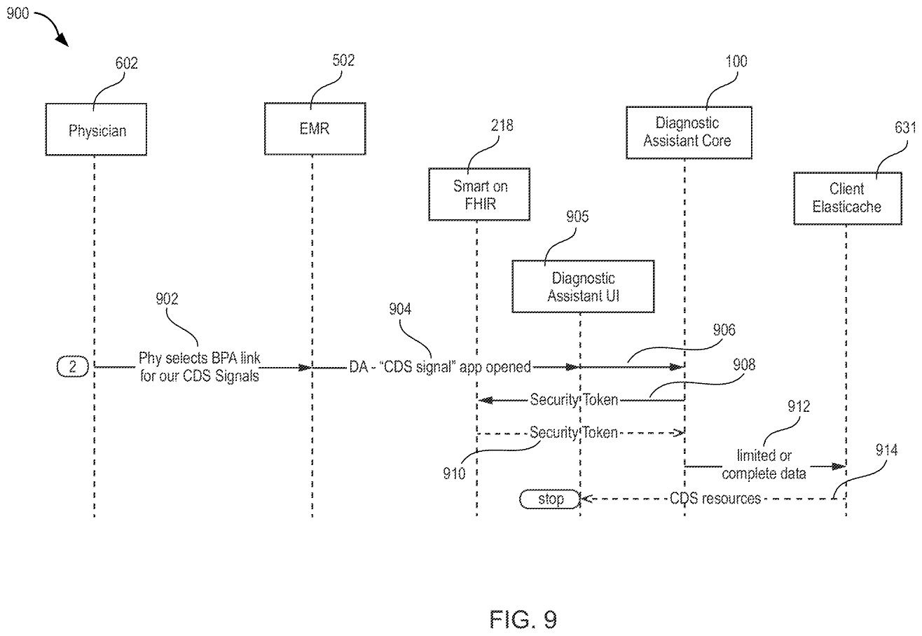

FIG. 9 is a swim-lane diagram 900 of data flow with respect to the clinical trial context of the diagnostic assistant tool 100 according to some embodiments of the present disclosure. While described in a particular order, the actions and operations described with respect to the swim-lane diagram 900 can be performed in any other suitable order including at least partially substantially contemporaneously. In some embodiments, the swim-lane diagram 900 begins with the medical professional 602, such as a physician or other suitable medical professional, at action 902 requesting resources relating to a patient. In some embodiments, the resources can include any suitable components of a medical history of the patient, eligibility considerations of the patient with respect to one or more clinical trials, and the like. The EMR system 502 can receive the request from the action 902, and the EMR system 502 can, at action 904, open a CDS application for executing the request. For example, the EMR system 502 may communicate with, or otherwise initialize, a diagnostic assistant UI 905 for executing the request. Additionally, the diagnostic assistant UI 905 can initialize the diagnostic assistant tool 100 at action 906.

The diagnostic assistant tool 100 can generate a security token at action 908 and can transmit the security token to the client security server 218 for authentication. The client security server 218 can generate and transmit an additional security token to the diagnostic assistant tool 100 at action 910. In some embodiments, the request for resources may be determined to be authentic if the security token and the additional security token correspond to one another, are identical, etc. The diagnostic assistant tool 100, in response to authenticating the request, can transmit data relating to the patient to the client elasticache 631 and/or any other suitable computing device or component thereof at action 912. And, at action 914, the client elasticache 631 can provide the requested resources to the diagnostic assistant tool 100. In some embodiments, the requested resources provided to the diagnostic assistant tool 100 can be output or otherwise provided by the diagnostic assistant tool 100, such as via the diagnostic assistant UI 905, to the medical professional 602.

In one such example, the diagnostic assistant tool 100 transmits a patient lookup request to the client elasticache 631, and the client elasticache returns data related to the patient. In another such example, such as subsequent to the previously described example, the diagnostic assistant tool 100 can transmit the data relating to the patient to the client elasticache 631, and the client elasticache 631 can transmit a set of clinical trials for which the patient is eligible. Other suitable examples are possible.

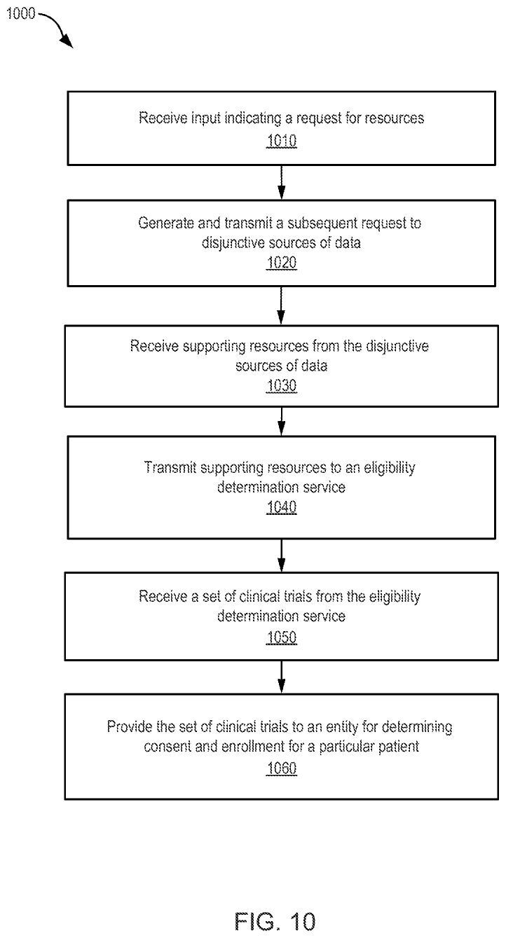

FIG. 10 is a flowchart of a process 1000 for determining clinical trial applicability and consent using the diagnostic assistant tool 100 according to some embodiments of the present disclosure. At block 1010, the diagnostic assistant services 504 receives a request for resources. In some embodiments, the medical professional 602 transmits the request for resources to the diagnostic assistant tool 100, such as via the diagnostic assistant UI 905, and the diagnostic assistant tool 100 transmits the request to the diagnostic assistant services 504. The request for resources may include a request for a set of clinical trials for which the patient is eligible, though the request for resources can include a request for any other suitable resources such as data relating to the patient, etc.

At block 1020, the diagnostic assistant services 504 generates and transmits a request to disjunctive sources of data. In some embodiments, the disjunctive sources of data include the EMR system 502, the provider database 152, the other sources of disjunctive data 512, and/or any other suitable disjunctive sources of data that may store or otherwise manage data relating to the patient. The request may include a request for support resources that include data relating to the patient included in the disjunctive sources of data. For example, neither the medical professional 602 nor the diagnostic assistant services 504 may include enough (or any) data about the patient for determining whether the patient is eligible for one or more clinical trials. The diagnostic assistant services 504 can generate the request for the support resources by transmitting the patient demographic and entity contextual information, and other suitable information, to each of the disjunctive sources of data. The request may cause one or more of the disjunctive sources of data to perform a patient lookup in a respective database.

At block 1030, the diagnostic assistant services 504 receives support resources from one or more of the disjunctive sources of data. The support resources can include data relating to the patient. In some embodiments, the support resources include medical history information, such as medical conditions, prescribed medications, demographic information, test results, historical visits to medical professionals, and the like, about the patient. The diagnostic assistant services 504 can receive the support resources from the EMR system 502, the provider database 152, the other sources of disjunctive data 512, or any suitable combination thereof. In some embodiments, the diagnostic assistant services 504 de-duplicates the support resources such that a mode of each distinct data point included in the support resources is one regardless of a number of times the data point is included in the EMR system 502, the provider database 152, and the other sources of disjunctive data 512.

At block 1040, the diagnostic assistant services 504 transmits the support resources to an eligibility determination service. In some embodiments, the eligibility determination services are or otherwise include the CQL services 806. The diagnostic assistant services 504 can transmit at least a portion of the support resources to the eligibility determination service. In one such example, the diagnostic assistant services 504 redacts a portion, such as personally identifiable information of the patient, of the support resources prior to transmitting the support resources to the eligibility determination service.

At block 1050, the diagnostic assistant services 504 receives a set of clinical trials from the eligibility determination service. In response to receiving the support resources, the eligibility determination service can generate the set of clinical trials such that each clinical trial included in the set of clinical trials includes parameters corresponding to the support resources. For example, if the support resources indicate that the patient experiences heart disease, then the eligibility determination service may identify one or more clinical trials for which individuals with heart disease may be eligible. In some embodiments, the support resources indicate a combination of conditions or other suitable parameters associated with the patient. Accordingly, the eligibility determination service can compare the support resources to the parameters of available clinical trials identified by the eligibility determination service. The eligibility determination service can determine the set of clinical trials, which may include zero, one, two, three, or more clinical trials, for which the patient may be eligible based on the comparison, and the eligibility determination service can transmit the set of clinical trials to the diagnostic assistant services 504.

At block 1060, the diagnostic assistant services 504 provides the set of clinical trials to the medical professional 602 for facilitating a determination of whether the patient is eligible for, and consents to, one or more clinical trials included in the set of clinical trials. The diagnostic assistant services 504 can receive the set of clinical trials and can provide, for example by displaying the set of clinical trials via the diagnostic assistant UI 905, the set of clinical trials for the medical professional 602. The medical professional 602 can use the provided set of clinical trials to make a decision regarding whether the patient is eligible for a particular clinical trial. In response to determining that the patient is eligible for the particular clinical trial, and in response to receiving consent from the patient to enroll the patient in the particular clinical trial, the medical professional 602 can input the consent for enrollment into the diagnostic assistant tool 100. For example, the medical professional 602 uploads or otherwise inputs the consent of the patient into the diagnostic assistant UI 905. Subsequently, the diagnostic assistant tool 100 can transmit the consent and enrollment data relating to the patient and the particular clinical trial to the QTR database 810. In some embodiments, the diagnostic assistant tool 100 redacts at least a portion, such as a subset of personally identifiable information, etc., of the consent and enrollment data relating to the patient prior to transmitting the data to the QTR database 810.

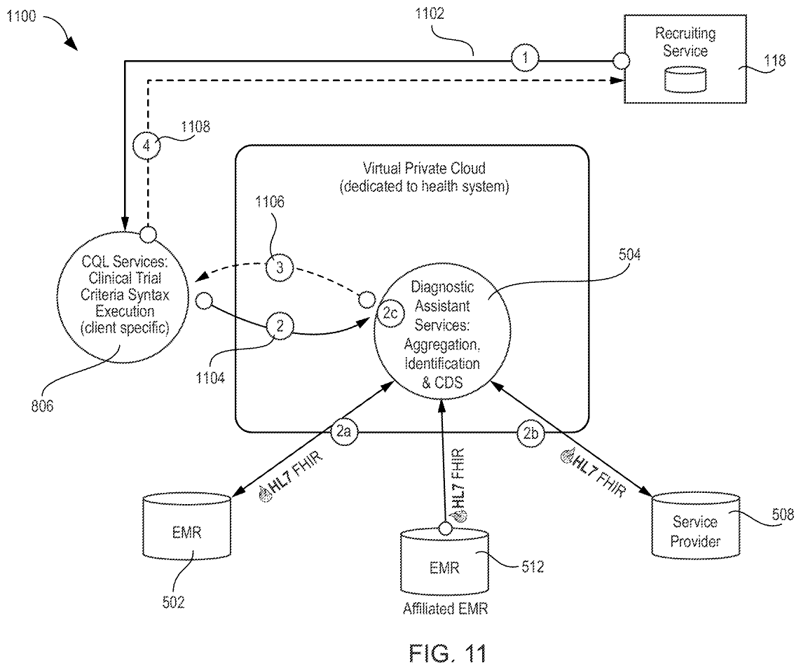

FIG. 11 is a block diagram of data flow with respect to a batch-processed clinical trial context of the diagnostic assistant tool 100 according to some embodiments of the present disclosure. The data flow diagram 1100 can represent a flow of data with respect to a medical professional triggering a query relating to a particular clinical trial. For example, the medical professional can search, for example using the QTR dashboard 118, for the particular clinical trial and can use the QTR dashboard 118 to submit the query or otherwise initialize the flow of data through the data flow diagram 1100. In other embodiments, data flow through the data flow diagram 1100 can be otherwise suitably initiated including automatically upon initiating the diagnostic assistant tool 100, etc. Additionally, data exchange and flow through the data flow diagram 1100 may be carried out using FHIR query and/or any other suitable protocol(s).

In some embodiments, a first data exchange 1102 is completed between the QTR dashboard 118 and the CQL services 806, which may include one or more eligibility determination services that can determine eligibility of a patient participating in the particular clinical trial. The QTR dashboard 118 can transmit the query to the CQL services 806, which initiates a batch process. In some embodiments, the batch process involves a second data exchange 1104 between the CQL services 806 and the diagnostic assistant services 504. The second data exchange 1104 can involve the CQL services 806 transmitting information relating to the particular clinical trial to the diagnostic assistant services 504. In some embodiments, the information relating to the particular clinical trial includes parameters that can be used to determine whether the patient is eligible to participate in the particular clinical trial.

The diagnostic assistant services 504 can execute the batch process initiated by the CQL services 806. For example, the diagnostic assistant services 504 can query the EMR system 502, the provider database 152, the other sources of disjunctive data 512, and/or any other suitable database for receiving data relating to a set of patients. In some embodiments, the batch process involves requesting, from the EMR system 502, the provider database 152, and the other sources of disjunctive data 512, data, including demographic information, medical history data, and the like, about the set of patients. Accordingly, the diagnostic assistant services 504 can submit queries to each of the EMR system 502, the provider database 152, and the other sources of disjunctive data 512 for gathering the data about the set of patients. Each of the EMR system 502, the provider database 152, and the other sources of disjunctive data 512 can transmit a set of data to the diagnostic assistant services 504. In one such example, the EMR system 502 can transmit a first set of data to the diagnostic assistant services 504, the provider database 152 can transmit a second set of data to the diagnostic assistant services 504, and the other sources of disjunctive data 512 can transmit a third set of data to the diagnostic assistant services 504, though other suitable amounts of sets of data can be transmitted by the EMR system 502, the provider database 152, and/or the other sources of disjunctive data 512.

In some embodiments, the diagnostic assistant services 504 receives the sets of data from the EMR system 502, the provider database 152, and/or the other sources of disjunctive data 512 and aggregates the sets of data into a set of patient data. The diagnostic assistant services 504 can de-duplicate the data included in the set of patient data. For example, the diagnostic assistant services 504 can perform a search through the set of patient data and adjust the set of patient data such that a mode of each data point in the set of patient data is one regardless of an amount of times that the data point exists in the first set of data, the second set of data, and the third set of data. Additionally, the data points included in the set of patient data can be enhanced, extracted, and/or added to a client elasticache, such as the client elasticache 631. In response to generating and adjusting the set of patient data, the diagnostic assistant services 504 can perform a third data exchange 1106 by transmitting at least a subset of the set of patient data. For example, the diagnostic assistant services 504 can redact at least a portion of the set of patient data prior to transmitting the set of patient data to the CQL services 806. The CQL services 806 can receive the set of patient data and can determine a set of patients, based on the set of patient data, which may be eligible for the particular clinical trial. Accordingly, the CQL services 806 can perform a fourth data exchange 1108 by transmitting the set of patients to the QTR dashboard 118 for the medical professional 602 to use in making one or more decisions about enrolling or requesting consent from one or more patients.

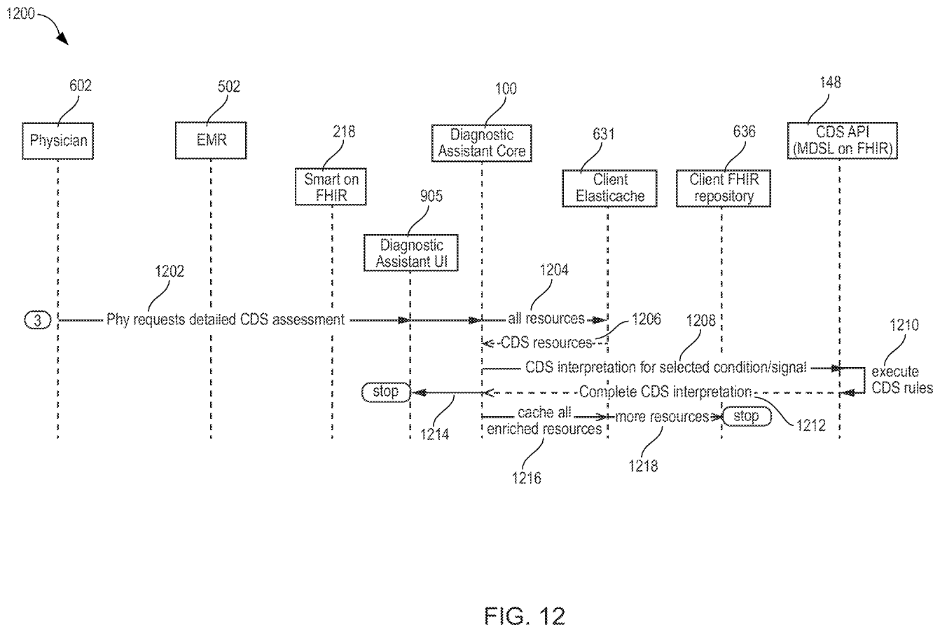

FIG. 12 is a swim-lane diagram 1200 of data flow with respect to the batch-processed clinical trial context of the diagnostic assistant tool 100, according to some embodiments of the present disclosure. While described in a particular order, the actions and operations described with respect to the swim-lane diagram 1200 can be performed in any other suitable order including at least partially substantially contemporaneously. In some embodiments, the swim-lane diagram 1200 begins at action 1202 in which the medical professional 602 requests resources. The medical professional 602 can request resources with respect to a particular clinical trial and/or any other suitable types of resources or data. In some embodiments, the action 1202 can be executed automatically, for example upon initiating the diagnostic assistant tool 100, etc. In some embodiments, the medical professional 602 transmits the request by inputting the request for the resources in the diagnostic assistant UI 905, which can, in turn, transmit the request to the diagnostic assistant tool 100. At action 1204, the diagnostic assistant tool 100 transmits resources to the client elasticache 631. In some embodiments, the request includes a set of resources that can include a type of the particular clinical trial, parameters of the particular clinical trial, and the like.

The client elasticache 631 can return CDS resources to the diagnostic assistant tool 100 at action 1206. In some embodiments, the CDS resources can include data relating to a set of patients in which the data was received by one or more of the EMR system 502, the provider database 152, or the other sources of disjunctive data 512. Additionally, the CDS resources can include data, such as medical history, demographic data, and the like, about the set of patients. Additionally, at action 1208, the diagnostic assistant tool transmits the received resources from the client elasticache 631 to the CDS API 148 for interpretation. For example, the CDS API 148 can execute CDS rules with respect to the transmitted resources at action 1210. In some embodiments, the interpretation may involve determining whether one or more of the patients, the data for whom is included in the transmitted data, is eligible for the particular clinical trial. Accordingly, the CDS API 148 at action 1212 can generate and transmit, to the diagnostic assistant tool 100, the CDS interpretation of the transmitted data relating to the set of patients.

In some embodiments, the diagnostic assistant tool 100 transmits the CDS interpretation to the diagnostic assistant UI 905 for providing the CDS interpretation to the medical professional 602 (e.g., via display on the diagnostic assistant UI 905) at action 1214. Additionally, and similar to the action 648 and the action 650, at action 1216, the CDS interpretation and associated resources are cached, for example in the client elasticache 631, and at action 1218, the cached resources are stored in the client FHIR repository 636.

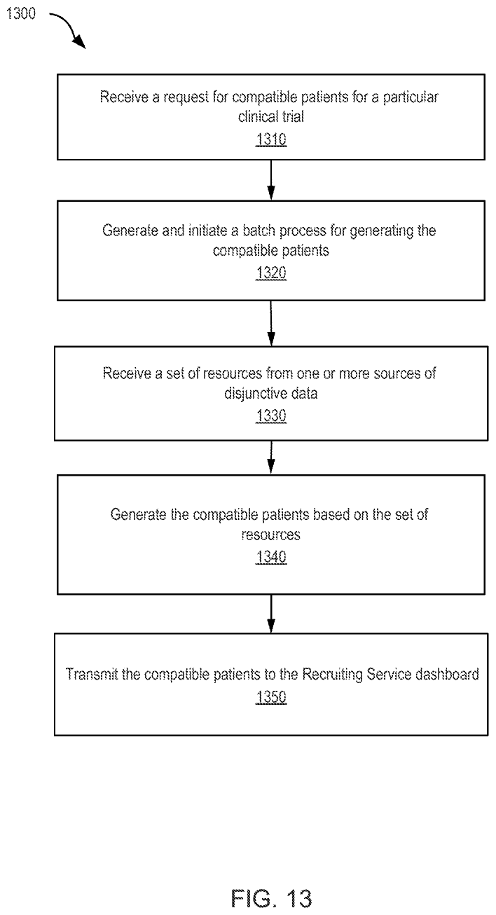

FIG. 13 is a flow chart of a process 1300 for determining clinical trial applicability using the diagnostic assistant tool 100, according to some embodiments of the present disclosure. At block 1310, the CQL services 806 receives a request for compatible patients for a particular clinical trial. The request may be input into the QTR dashboard 118, for example, by the medical professional 602. In some embodiments, the compatible patients includes or is intended to include a set of patients that each are eligible to participate in the particular clinical trial. Accordingly, the medical professional 602 may input data, such as a type, parameters, and the like, relating to the particular clinical trial into the QTR dashboard 118, and the QTR dashboard 118 can transmit the request to the CQL services 806. In some embodiments, the request for compatible patients can initiate a batch process. The batch process can be run in response to the query request of off hours to reduce computational demand during peak times. The batch process can run either patient by patient or a functional language can be applied against a full list of patient resources to reduce computational demands.

At block 1320, the CQL services 806 generates and triggers the batch process for generating the compatible patients. The CQL services 806 ingests the request for the compatible patients and can generate the batch process, which may involve gathering or otherwise receiving data relating to a set of patients. The CQL services 806 can trigger the batch process request by transmitting the batch process request to the diagnostic assistant services 504. In some embodiments, the batch process request is a batch process since data for more than one patient is queried.

In some embodiments, the diagnostic assistant services 504 executes the batch process request by transmitting an updated request to one or more sources of disjunctive data. For example, the diagnostic assistant services 504 can query the EMR system 502, the provider database 152, the other sources of disjunctive data 512, and/or any other suitable database for receiving data relating to a set of patients. In some embodiments, the batch process involves requesting, from the EMR system 502, the provider database 152, and the other sources of disjunctive data 512, data, including demographic information, medical history data, and the like, about the set of patients. Accordingly, the diagnostic assistant services 504 can submit queries to each of the EMR system 502, the provider database 152, and the other sources of disjunctive data 512 for gathering the data about the set of patients. Each of the EMR system 502, the provider database 152, and the other sources of disjunctive data 512 can transmit a set of data to the diagnostic assistant services 504. In one such example, the EMR system 502 can transmit a first set of data to the diagnostic assistant services 504, the provider database 152 can transmit a second set of data to the diagnostic assistant services 504, and the other sources of disjunctive data 512 can transmit a third set of data to the diagnostic assistant services 504, though other suitable amounts of sets of data can be transmitted by the EMR system 502, the provider database 152, and/or the other sources of disjunctive data 512.

In some embodiments, the diagnostic assistant services 504 receives the sets of data from the EMR system 502, the provider database 152, and/or the other sources of disjunctive data 512 and aggregates the sets of data into a set of patient data. The diagnostic assistant services 504 can de-duplicate the data included in the set of patient data. For example, the diagnostic assistant services 504 can perform a search through the set of patient data and adjust the set of patient data such that a mode of each data point in the set of patient data is one regardless of a number of times that the data point exists in the first set of data, the second set of data, and the third set of data.

At block 1330, the CQL services 806 receives a set of resources from one or more sources of disjunctive data. The set of resources can be or otherwise include the set of patient data. For example, the diagnostic assistant services 504 can generate the set of patient data, which includes data, such as medical history, demographic data, and the like, about various patients, and the diagnostic assistant services 504 can transmit at least a portion of the set of patient data to the CQL services 806. In some embodiments, the diagnostic assistant services 504 redacts at least a portion of the set of patient data prior to transmitting the set of patient data to the CQL services 806. The set of patient data can include data originating from the EMR system 502, the provider database 152, the other sources of disjunctive data 512, and/or any other suitable source of patient data accessible by the diagnostic assistant services 504.

At block 1340, the CQL services 806 generates, based on the set of resources, the compatible patients for the particular clinical trial. In some embodiments, the CQL services 806 compares the set of patient data, such as the set of resources, to the data about the particular clinical trial. For example, the CQL services 806 can compare medical history, demographic data, and the like relating to patients included in the set of patient data to parameters of the particular clinical trial to determine whether one or more patients associated with the set of patient data is eligible to participate in the particular clinical trial. In some embodiments, and in response to the comparison, the CQL services 806 identifies one or more patients that may be eligible for the particular clinical trial. Accordingly, the CQL services 806 can generate, based on the identified one or more patients, the compatible patients.

At block 1350, the CQL services 806 transmits the compatible patients for the particular clinical trial to the QTR dashboard 118. In some embodiments, the CQL services 806 at least partially redacts or partially withholds data relating to the compatible patients. For example, some demographic information, personal information, and the like may be excluded from the compatible patients prior to transmitting the compatible patients to the QTR dashboard 118. In some embodiments, the CQL services 806 transmits the compatible patients to the QTR dashboard 118 for facilitating one or more decisions by the medical professional 602. For example, based on the compatible patients provided to the medical professional 602 by the QTR dashboard 118, or other suitable component or computing device, the medical professional 602 can decide whether a patient or a particular set of patients is eligible for the particular clinical trial and can solicit consent and/or enrollment information from the patient or the particular set of patients.

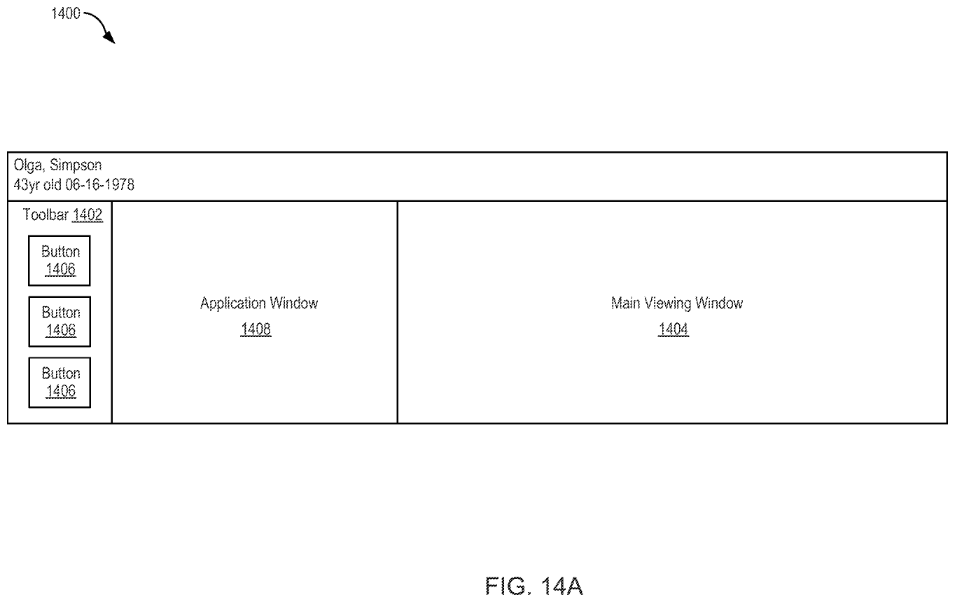

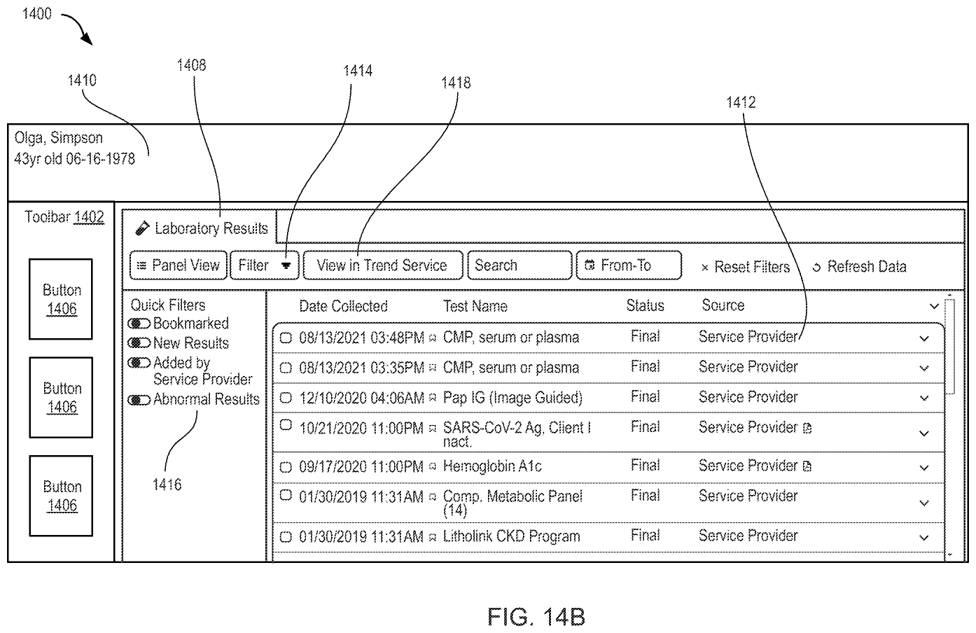

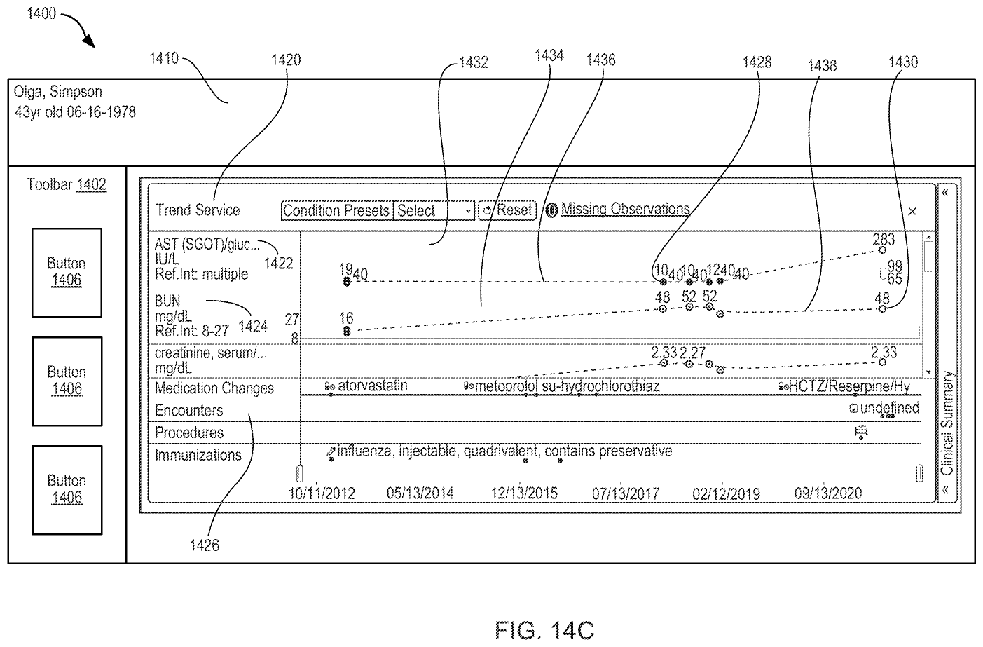

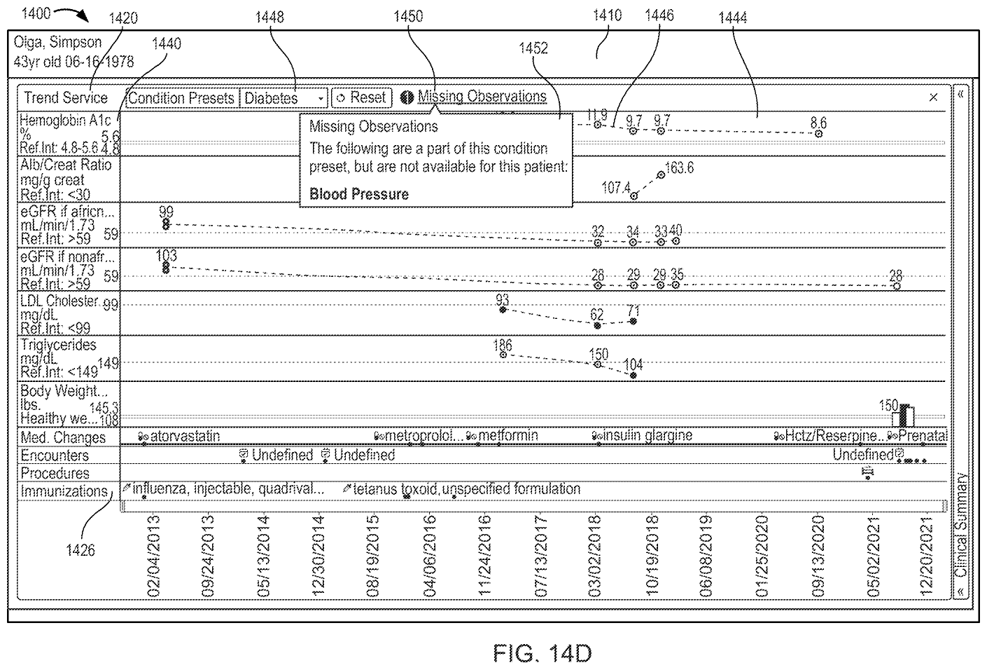

FIG. 14A is an example of a user interface 1400 displaying information and for launching the diagnostic assistant tool 100, according to some embodiments of the present disclosure. In some embodiments, the user interface 1400 is generated and provided by the EMR system 502 or other suitable medical records system, to which the medical professional 602, such as a physician or other suitable medical professional, can login to view patient information. For example, the main viewing window 1404 can display information from the EMR system 502 relating to a patient visiting the medical professional 602. As illustrated in FIG. 14A, the user interface 1400 includes a toolbar 1402 and a main viewing window 1404. The toolbar 1402 can include with one or more interactive buttons 1406 that, when interacted with by the medical professional 602, can execute one or more tasks relating to the EMR system 502. For example, the main viewing window 1404 can display various results in response to interaction with the one or more interactive buttons 1406 of the toolbar 1402. For example, selecting a button (e.g., one of the interactive buttons 1406) can cause the diagnostic assistant tool 100 to launch within an application window 1408.

In some embodiments, the toolbar 1402 includes the one or more interactive buttons 1406 that, in response to receiving input from the medical professional 602, can cause the user interface 1400 to display the application window 1408. The interactive buttons 1406 can be associated with different third-party applications that can be accessed within the user interface 1400 via the interactive buttons 1406. Upon receiving input via the interactive buttons 1406, the user interface 1400 can generate and display the application window 1408, which includes one or more interactive elements corresponding to applications that was accessed. For example, in response to receiving selection of one of the interactive buttons 1406 corresponding to the diagnostic assistant tool 100, the EMR system 502 can launch the diagnostic assistant tool 100 within the application window 1408. In some embodiments, the user interface 1400 or other suitable component of the EMR system 502 can automatically launch the diagnostic assistant tool 100 in response to the medical professional 602 logging in, for example, to the EMR system 502 for a visit by the patient. The medical professional 602 can interact with the elements within the application through the application window.

In some embodiments, an application can be loaded within the application window 1408 in a manner in which the data displayed within the application window 1408 is not accessible by the client device displaying the user interface 1400. Similarly, the data provided within the application window 1408 can be continuously updated such that the data is provided in real-time or otherwise substantially contemporaneously. The information within the application window 1408 can be provided through an application such as the diagnostic assistant tool 100 in any combination of methods. For example, the application window 1408 can load the diagnostic assistant tool 100 using an iframe. Therefore, the information provided by the diagnostic assistant tool 100 within the application window 1408 can be securely controlled and updated by the diagnostic assistant services 504 while the remaining portions, such as main viewing window 1404, of the user interface 1400 can be provided and controlled by the local or host system such as the EMR 502.

FIG. 14B is an example of a user interface 1400 displaying the diagnostic assistant tool 100, or any suitable feature thereof, according to some embodiments of the present disclosure. As illustrated, the user interface 1400 includes patient identification information 1410 and an application loaded within the application window 1408. In some embodiments, an application displaying laboratory results can be displayed within the application window 1408. The patient identification information 1410 includes a name of the patient that is visiting the medical professional 602, a date of birth of the patient, a medical record number of the patient, etc. The patient identification information 1410 can include any other suitable information that can be used to, or in conjunction with, identifying the patient. The application window 1408 is displaying resources associated with the patient. For example, and as illustrated, the application window 1408 includes a resource list 1412, a filter selection tool 1414, a quick filter list 1416, and a trend feature link 1418.

The user interface 1400 can generate a list of resources upon the diagnostic assistant tool 100 launching, and the user interface 1400 can generate and display the resource list 1412 for providing resources, such as data relating to the patient. As illustrated, the resource list 1412 includes a list of entries that includes test records of the patient, though other suitable resources can be received by the user interface 1400 and displayed via the resource list 1412. In some embodiments, input can be received via the filter selection tool 1414 to filter or otherwise adjust the resources displayed via the resource list 1412. For example, the filter selection tool 1414 can allow the medical professional 602 to select a predetermined medical condition filter, and the predetermined medical condition filter can cause the user interface 1400 to adjust the resource list 1412 to display resources corresponding to the medical condition represented by the predetermined medical condition filter. The filter selection tool 1414 can include any other suitable types of filters, including filters customized or otherwise generated via input from the medical professional 602, for adjusting the resources of the patient displayed with respect to the resource list 1412.

In some embodiments, the application window 1408 includes the quick filter list 1416 for allowing the medical professional 602 to quickly adjust the resources displayed via the application window 1408. For example, and as illustrated, the quick filter list 1416 includes a set of interactive elements for selecting to view resources about the patient that are bookmarked, that are new, that originate from a certain source of data, and that are considered abnormal. Other suitable quick filters, including customized filters or filters otherwise generated via input from the medical professional 602, are possible. Additionally or alternatively, the application window 1408 includes the trend feature link 1418 that, in response to receiving input, causes the user interface 1400 to generate and output a trend capability that can display one or more trends with respect to the resources included in the resource list 1412. The application window 1408 can include any other suitable features, applications, and the like for providing the resources about the patient to the medical professional 602.

FIG. 14C is an example of a user interface 1400 displaying an application with features of a trend capability 1420 of the diagnostic assistant tool 100 according to some embodiments of the present disclosure. In some embodiments, the user interface 1400 is generated in response to the medical professional 602 interacting with the trend feature link 1418 provided on the user interface 1400 of FIG. 14B. As illustrated, the user interface 1400 includes the patient identification information 1410 that includes the name of the patient that is visiting the medical professional 602, the date of birth of the patient, the medical record number of the patient, etc. Additionally, the user interface 1400 includes the trend capability 1420 of the diagnostic assistant tool 100. The trend capability 1420 can visually provide the resources requested by the medical professional 602. For example, the trend capability 1420 can graphically plot the resources and provide the plot for aiding the medical professional 602 in making one or more decisions.

As illustrated, the trend capability 1420 includes a first resource 1422, a second resource 1424, and ancillary data 1426. The first resource 1422 includes test results for a level of glucose for the patient, and the second resource 1424 includes test results for blood urea nitrogen (BUN) for the patient. Additionally, the ancillary data 1426 includes information about the patient relating to medication changes, encounters with the medical professional 602 or other medical professionals, medical procedures undergone by the patient, and immunizations administered to the patient. The first resource 1422, the second resource 1424, and the ancillary data 1426 can include any other suitable data, including customized data based on input received from the medical professional 602, relating to the patient.

In some embodiments, each resource displayed via the trend capability 1420 includes one or more data points. For example, the first resource 1422 includes six data points, which may include a first data point 1428, and the second resource 1424 also includes six data points, which may include a second data point 1430. In other examples, the first resource 1422 and the second resource 1424 may include different numbers (e.g., less than six or more than six) of data points. As illustrated in FIG. 14C, the data points of the first resource 1422 are plotted and displayed on a first plot 1432, and the data points of the second resource 1424 are plotted and displayed on a second plot 1434, which may be displayed vertically, or otherwise suitably, offset from the first plot 1432 but temporally aligned with the first plot 1432. Additionally, the first data point 1428 is solid, and the second data point 1430 is hollow. In some embodiments, a solid data point indicates that an origin of the respective data point is from the provider of the diagnostic assistant tool 100. Additionally, a hollow data point indicates that an origin of the respective data point is not from the provider of the diagnostic assistant tool 100.

In some embodiments, the data points of each of the first resource 1422 and the second resource 1424 can be connected to form a trend line. As illustrated, the data points on the first plot 1432 corresponding to the first resource 1422 are connected via a first trend line 1436, and the data points on the second plot 1434 corresponding to the second resource 1424 are connected via a second trend line 1438. In some embodiments, the first trend line 1436 and the second trend line 1438 can directly connect each of the data points of the first resource 1422 and the second resource 1424, respectively. In other embodiments, the first trend line 1436, the second trend line 1438, or a combination thereof can be continuous curves that represent a general trend of the data points of the first resource 1422 or the second resource 1424, respectively. Additionally, while not illustrated with respect to FIG. 14C, the first plot 1432, the second plot 1434, and/or any other suitable plot generated and displayed via the user interface 1400 can include a displayed range of acceptable values corresponding to each of the displayed resources.

FIG. 14D is an example of a user interface 1400 displaying additional features of the trend capability 1420 of the diagnostic assistant tool 100 according to some embodiments of the present disclosure. As illustrated, the user interface 1400 includes the patient identification information 1410 that includes the name of the patient that is visiting the medical professional 602, the date of birth of the patient, the medical record number of the patient, etc. Additionally or alternatively, the user interface 1400 can include the trend capability 1420 of the diagnostic assistant tool 100. The trend capability 1420 can visually provide the resources requested by the medical professional 602. For example, the trend capability 1420 can graphically plot the resources and provide the plot for aiding the medical professional 602 in making one or more decisions.

The trend capability 1420 can include a set of resources, which may include a third resource 1440, relating to the patient. Each resource of the set of resources can include data points, which may include a third data point 1442, that are plotted on corresponding plots, such as a third plot 1444, and that are connected via trend lines, such as a third trend line 1446. Additionally or alternatively, the trend capability 1420 includes a condition selector 1448. The condition selector 1448 can include a set of interactive elements, which can be displayed via a drop-down menu or other suitable manner, that correspond to different medical conditions. For example, the condition selector 1448 can include an interactive element corresponding to diabetes, illustrated as selected in FIG. 14D. Selecting the diabetes interactive element may cause the resources, such as the first resource 1422, the second resource 1424, and/or the third resource 1440, etc., to be adjusted. For example, selecting the diabetes interactive element may cause resources not associated with diabetes to be excluded from the trend capability 1420.

Additionally, the trend capability 1420 includes a missing observation feature 1450 subsequent to a condition being selected from the condition selector 1448. The missing observation feature 1450 can include an interactive element that, when selected, can display a missing information notification 1452. In some embodiments, the missing information notification 1452 includes information that indicates, for example to the medical professional 602, that one or more observations is missing from the resources displayed via the trend capability 1420. For example, if a test result, such as blood pressure or other suitable test result, associated with the selected condition is missing from the set of displayed resources, then the user interface 1400 can generate the missing information notification 1452. In some embodiments, the medical professional 602 can use the missing information notification 1452 to make one or more decisions with respect to the patient. For example, and relating to the previous example, the medical professional 602 can recommend that the patient undergo a blood pressure test to remedy the missing information. The medical professional 602 can otherwise suitably use the user interface 1400 (and/or any of the previously described user interfaces) to make one or more decisions with respect to the patient.

As described above with respect to the user interface 1400, various parameters exist within the respective user interface. Some parameters, for example the filters from the filter selection tool 1414, the interactive elements from the condition selector, and the like, may be associated with medical conditions. The parameters, such as which resources correspond to particular medical conditions, may be set by or based on guidance from professional or governmental associations. In some embodiments, the pre-set parameters may be adjusted by the medical professional 602 for ease of viewability, for increased assistance in decision-making, or for any suitable purposes based on the discretion of the medical professional 602.

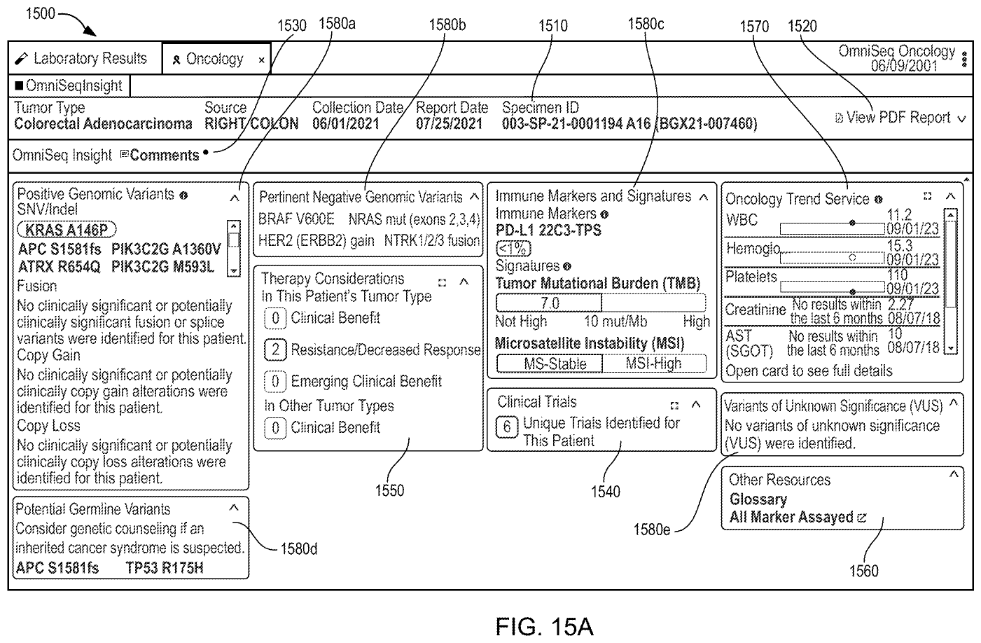

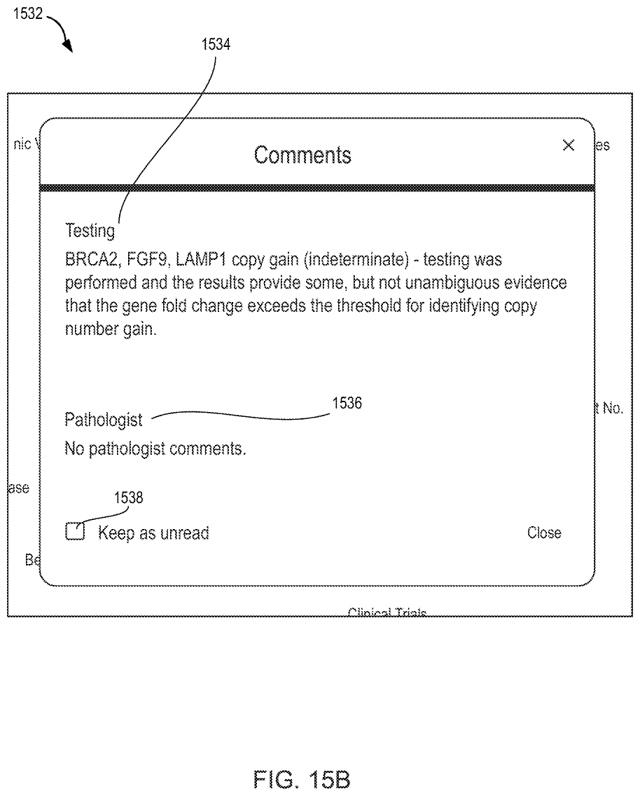

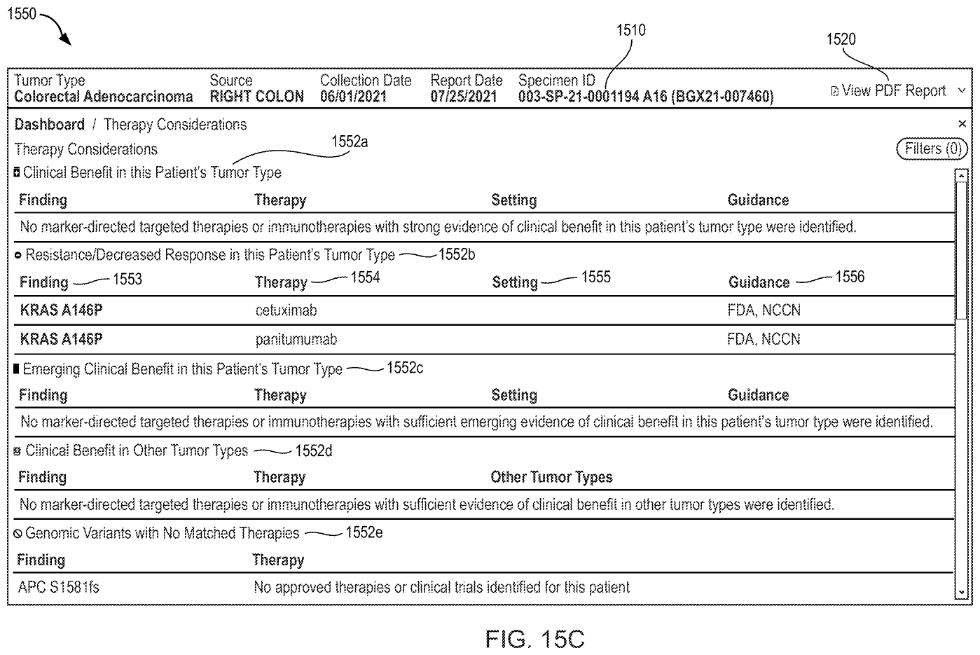

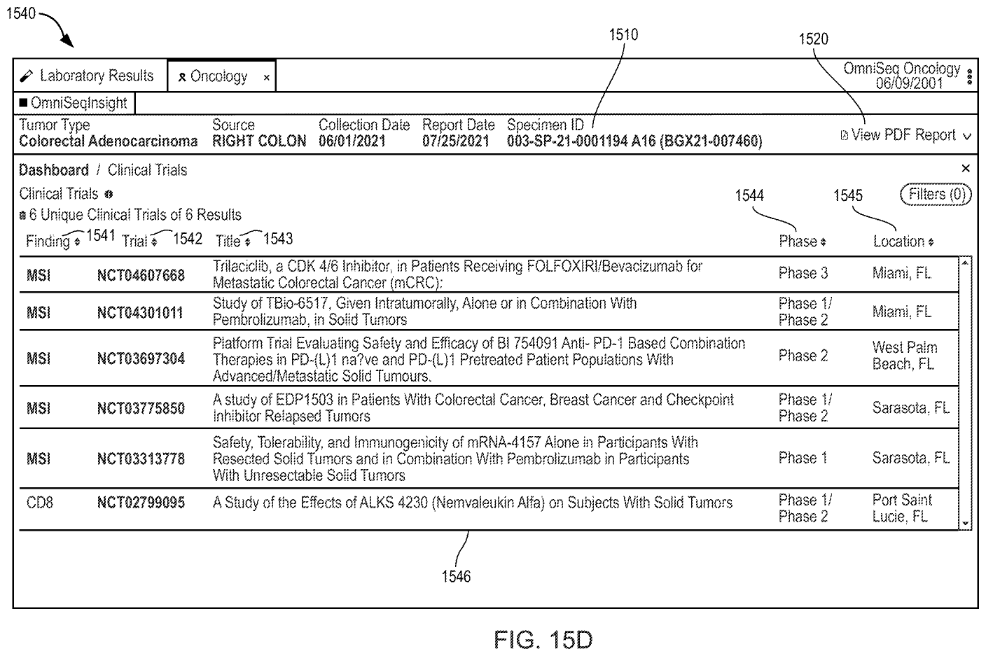

FIG. 15A is an example of a user interface displaying a digital dashboard 1500 of the diagnostic assistant tool of FIG. 1 according to some embodiments of the present disclosure. The digital dashboard 1500 can be an augmentation to original reports. While FIG. 15A depicts a digital dashboard 1500 associated with oncology, other discipline categories are available for the digital dashboard 1500 such as radiology, pathology, neurology, internal medicine, pediatrics, gastroenterology, cardiology, emergency medicine, immunology, surgery, etc. A physician can use the digital dashboard 1500 to access reported results, view patient specific clinical trials, medications, therapy considerations, etc. The digital dashboard 1500 can include a reference bar 1510, a comments link 1530, and several expandable cards. The reference bar 1510 can include information related to a diagnosis for a patient based on a laboratory result. For example, if a laboratory result identifies a tumor, the reference bar 1510 can indicate a type of tumor, a location in the body for a source of the tumor, a date of identification, specimen ID associated with the tumor, etc. The reference bar 1510 can also include a pdf report link 1520 that a physician can use to view an original report that the digital dashboard 1500 augments.

The comments link 1530 can open a comments card that can let the physician view narrative comments regarding testing for the patient. The expandable cards can include a clinical trials card 1540, a therapy considerations card 1550, an other resources card 1560, a SmartTrends card 1570, discipline specific cards 1580a-e, etc. Although there are five discipline specific cards depicted in FIG. 15A, the digital dashboard 1500 can include any number of discipline specific cards. Discipline specific cards 1580a-e for the digital dashboard 1500 associated with oncology can include a positive genomic variants card 1580a, a pertinent negative genomic variants card 1580b, an immune markers and signatures card 1580c, a potential germline variants card 1580d, and a variants of unknown significance card 1580e. The other resources card 1560 can include a glossary. The SmartTrends card 1570 can include interactive plots that allow the physician to view a timeline for various test results for the patient.

FIG. 15B is an example of a user interface displaying a comments card 1532 of the digital dashboard 1500 of FIG. 15A according to some embodiments of the present disclosure. The comments card 1532 can be accessed by selecting/clicking/activating the comments link 1530 of the digital dashboard 1500. The comments card 1532 can include a testing section 1534, a specialist comments section 1536, and a keep as unread toggle button 1538. The testing section 1534 can include a listing of testing performed on a patient as well as comments associated with possible interpretations of results. The specialist comments section 1536 can provide a designated space for a specialist (e.g., a pathologist, etc.) to share an opinion regarding test results. The unread toggle button 1538 can be selected to mark the comments card 1532 as unread, even if a physician has viewed the comments card 1532. A physician may choose to select the unread toggle button 1538 if the physician wishes to return to the comments card 1532 at a later time, for example, to check to see if a specialist has added comments in the specialist comments section 1536.

FIG. 15C is an example of a user interface displaying an expanded therapy considerations card 1550 of the digital dashboard 1500 of FIG. 15A according to some embodiments of the present disclosure. The therapy considerations card 1550 can specify/detail therapy considerations based on findings specific to results for a patient. The expanded therapy considerations card 1550 can include a reference bar 1510. The reference bar 1510 can be identical to the reference bar 1510 of the digital dashboard 1500. The reference bar 1510 can include information related to a diagnosis for a patient based on a laboratory result. For example, if a laboratory result identifies a tumor, the reference bar 1510 can indicate a type of tumor, a location in the body for a source of the tumor, a date of identification, specimen ID associated with the tumor, etc. The reference bar 1510 can also include a pdf report link 1520 that a physician can use to view an original report that the digital dashboard 1500 augments.

The expanded therapy considerations card 1550 can also include several categories 1552a-e of therapy considerations. The categories 1552a-e can be based on potential benefits of therapies and can be organized in row sections of the therapy considerations card 1550. Although five categories 1552a-e are shown in FIG. 15C any number of categories can be included on the therapy considerations card 1550. Each of the categories 1552a-e can include columns, such as a finding column 1553, a therapy column 1554, a setting column 1555, and a guidance 1556 column. Although four columns 1553-6 are shown in FIG. 15C, any number of columns can be included for each of the categories 1552a-e. As an example, a therapy considerations card 1550 for oncology can include a ‘resistance/decreased response in this patient's tumor type’ category 1552b. This category 1552b can include two identical entries in the finding column 1553: KRAS A146P. The category 1552b can also include two entries in the therapy column 1554: cetuximab and panitumumab. The setting column 1555 for this category 1552b can be blank, indicating that both of the therapies may not be associated with a particular setting. The guidance column 1556 can indicate authoritative bodies (e.g., National Comprehensive Cancer Network (NCCN), the Food and Drug Administration (FDA), etc.) that recommend each therapy.

FIG. 15D is an example of a user interface displaying an expanded clinical trials card 1540 of the digital dashboard 1500 of FIG. 15A according to some embodiments of the present disclosure. The expanded clinical trials card 1540 can provide additional details regarding clinical trials based on findings in data associated with a patient. The expanded clinical trials card 1540 can include a reference bar 1510. The reference bar 1510 can be identical to the reference bar 1510 of the digital dashboard 1500. The reference bar 1510 can include information related to a diagnosis for a patient based on a laboratory result. For example, if a laboratory result identifies a tumor, the reference bar 1510 can indicate a type of tumor, a location in the body for a source of the tumor, a date of identification, specimen ID associated with the tumor, etc. The reference bar 1510 can also include a pdf report link 1520 that a physician can use to view an original report that the digital dashboard 1500 augments.

The clinical trials card 1540 can further include a table 1546. The table 1546 can include several columns, including a finding column 1541, a trial column 1542, a title column 1543, a phase column 1544, and a location column 1545. For each entry in the finding column 1541, the table 1546 can indicate at least one clinical trial in the trial column 1542. The title for each identified clinical trial can be shown in the title column 1543. The phase that each trial is currently in can be shown in the phase column 1544. The location of each clinical trial can be shown in the location column 1545.

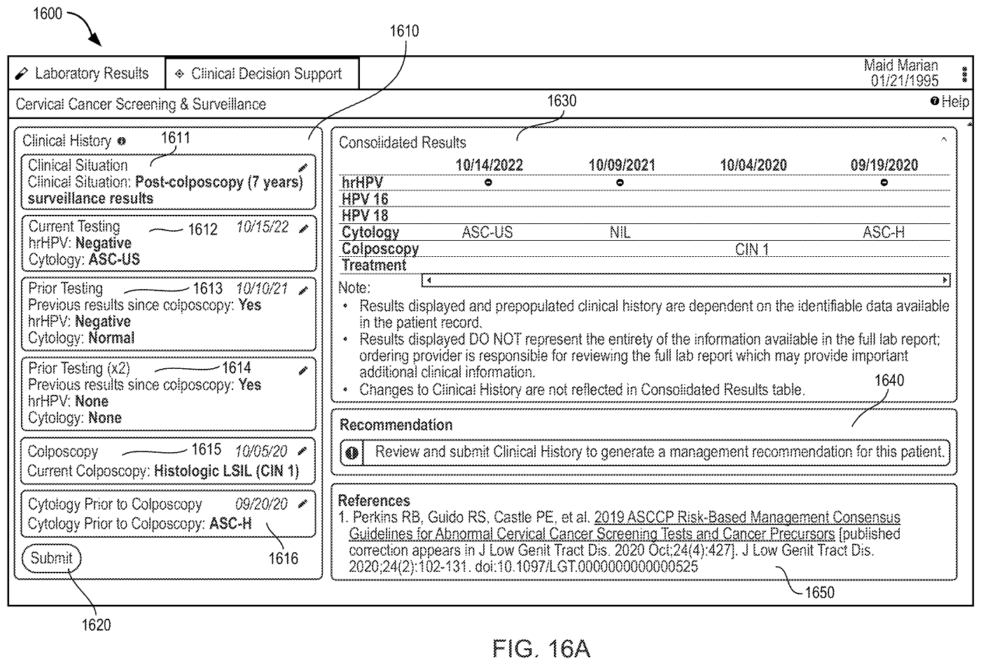

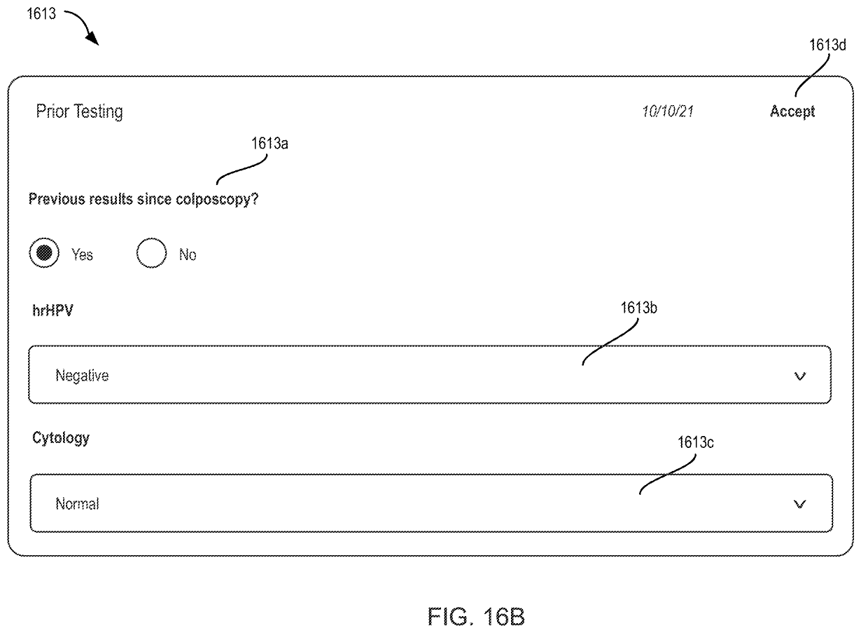

FIG. 16A is an example of a user interface displaying a clinical decision support (CDS) questionnaire 1600 for the diagnostic assistant tool of FIG. 1 according to some embodiments of the present disclosure. The CDS questionnaire 1600 can be used to help consider a clinical situation of context for a patient along with patient specific results to populate clinical recommendations along with risk scores. The questionnaire 1660 can be based in part on guidelines provided by professional organizations, such as the American Society for Colonoscopy and Cervical Pathology (ASCCP). The questionnaire 1660 can be prepopulated with responses based on known patient data and results, such as from data maintained in an EHR for the patient. In some examples, prepopulated responses can include an indicator. The indicator can assist a user in identifying which responses were prepopulated. The indicator can include information, such as a date or database source, regarding data that was used to prepopulate a response so that the user can identify source data that was used to prepopulate the response. A user can modify or add responses to include information that may not be available in the EHR. The questionnaire 1600 can be a dynamic interface. For example, a particular input to a first portion of the questionnaire 1600 can automatically determine a response to a second portion and the interface can automatically populate the second portion with an appropriate response.

The questionnaire 1600 can include a clinical history module 1610, a consolidated results module 1630, a recommendation module 1640, and a references module 1650. The clinical history module 1610 can include several expandable question cards 1611-6. For example, a questionnaire 1600 associated with cervical cancer screening and surveillance can include a clinical situation card 1611, a current testing card 1612, a prior testing card 1613, a prior testing (X2) card 1614, a colposcopy card 1615, and a cytology prior to colposcopy card 1616. The clinical history module 1610 can also include a submit button 1620. Once a user presses the submit button 1620, a CDS engine of the diagnostic assistant tool can generate a recommendation and populate the recommendation module 1640 with graphical elements associated with the recommendation. The reference module 1650 can be updated based on the generated recommendation. For example, additional references can be added to the reference module 1650 based on the generated recommendation or a page range of a reference can change based on the generated recommendation.

FIG. 16B is an example of a user interface displaying an expanded question card of the CDS questionnaire 1600 of FIG. 16A according to some embodiments of the present disclosure. For example, the expanded question card can be an expanded version of the prior testing card 1613 from FIG. 16. The expanded question card can include a question 1613a, such as a yes or no question. Depending on a selected answer to the question 1613a, the expanded question card can also include pull down menus, such as pull down menu 1613b and pull down menu 1613c. In some examples, the pull down menus may not be present (e.g., when an answer to question 1613a is no, etc.). The expanded question card can also include an accept button 1613d. When a user presses the accept button 1613d, the questionnaire 1600 can be updated with accepted selections to the question card and in some cases, other question cards can be populated based on the selections.

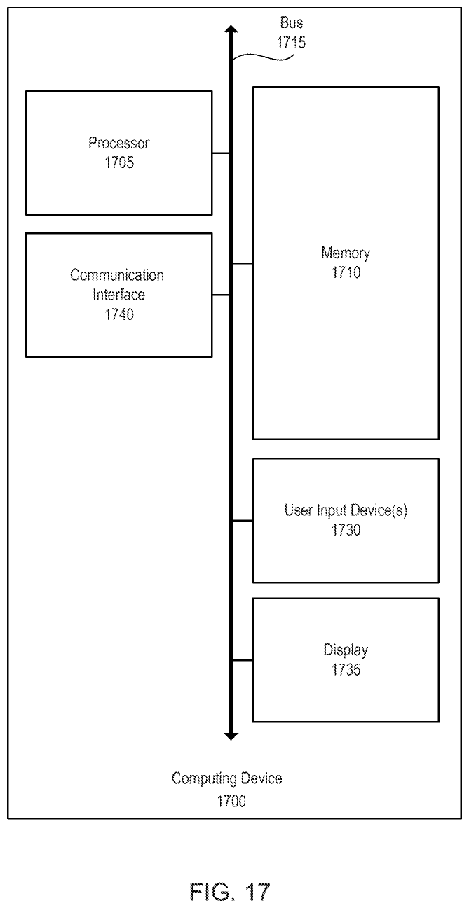

FIG. 17 is block diagram of a computing device 1700 for use with the diagnostic assistant tool 100 in accordance with various embodiments. The computing device 1700 includes a processor 1705, which is in communication with the memory 1710 and other components of the computing device 1700 using one or more communications buses 1715. The processor 1705 is configured to execute processor-executable instructions stored in the memory 1710 to perform one or more processes according to different examples, such as part or all of the examples of processes 700, 1000, and 1300 described respectively above with respect to FIGS. 7, 10, and 13. In some examples, the memory 1710 stores processor-executable instructions that provide the various components and their related functionality, as discussed above with respect to FIGS. 1-16B. The computing device 1700, for example, also includes one or more user input devices 1730, such as a keyboard, mouse, touchscreen, microphone, etc., to accept user input. The computing device 1700 also includes a display 1735 to provide visual output to a user such as a user interface.

The computing device 1700 also includes a communications interface 1740. In some examples, the communications interface 1740 may enable communications using one or more networks, including a local area network (“LAN”), a wide area network (“WAN”), such as the Internet, metropolitan area network (“MAN”), point-to-point or peer-to-peer connection, etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and systems herein are described in terms of software executing on various machines, the methods and systems may also be implemented as specifically configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor includes a computer-readable medium, such as a random-access memory (RAM) coupled to the processor. The processor executes computer-executable program instructions stored in memory, such as executing one or more computer programs. Such processors may comprise a microprocessor, a digital signal processor (DSP), an algorithm-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, which may store processor-executable instructions that, when executed by the processor, can cause the processor to perform methods according to this disclosure as carried out, or assisted, by a processor. Examples of non-transitory computer-readable medium may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

Although specific examples have been described, various modifications, alterations, alternative constructions, and equivalents are possible. Examples are not restricted to operation within certain specific data processing environments but are free to operate within a plurality of data processing environments. Additionally, although certain examples have been described using a particular series of transactions and steps, it should be apparent to those skilled in the art that this is not intended to be limiting. Although some flowcharts describe operations as a sequential process, many of the operations may be performed in parallel or concurrently. In addition, the order of the operations may be rearranged. A process may have additional steps not included in the figure. Various features and aspects of the above-described examples may be used individually or jointly.

Further, while certain examples have been described using a particular combination of hardware and software, it should be recognized that other combinations of hardware and software are also possible. Certain examples may be implemented only in hardware, or only in software, or using combinations thereof. The various processes described herein may be implemented on the same processor or different processors in any combination.

Where devices, systems, components or modules are described as being configured to perform certain operations or functions, such configuration may be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes may communicate using a variety of techniques including but not limited to conventional techniques for inter-process communications, and different pairs of processes may use different techniques, or the same pair of processes may use different techniques at different times.

Specific details are given in this disclosure to provide a thorough understanding of the examples. However, examples may be practiced without these specific details. For example, well-known circuits, processes, algorithms, structures, and techniques have been shown without unnecessary detail in order to avoid obscuring the examples. This description provides example examples only, and is not intended to limit the scope, applicability, or configuration of other examples. Rather, the preceding description of the examples will provide those skilled in the art with an enabling description for implementing various examples. Various changes may be made in the function and arrangement of elements.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific examples have been described, these are not intended to be limiting. Various modifications and equivalents are within the scope of the following claims.

In the foregoing specification, aspects of the disclosure are described with reference to specific examples thereof, but those skilled in the art will recognize that the disclosure is not limited thereto. Various features and aspects of the above-described disclosure may be used individually or jointly. Further, examples may be utilized in any number of environments and applications beyond those described herein without departing from the broader spirit and scope of the specification. The specification and drawings are, accordingly, to be regarded as illustrative rather than restrictive.

In the foregoing description, for the purposes of illustration, methods were described in a particular order. It should be appreciated that in alternate examples, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Where components are described as being configured to perform certain operations, such configuration may be accomplished, for example, by designing electronic circuits or other hardware to perform the operation, by programming programmable electronic circuits (e.g., microprocessors, or other suitable electronic circuits) to perform the operation, or any combination thereof.

While illustrative examples of the application have been described in detail herein, it is to be understood that the inventive concepts may be otherwise variously embodied and employed, and that the appended claims are intended to be construed to include such variations, except as limited by the prior art.

What is claimed is:

1. A system comprising:
a processor; and
a non-transitory computer-readable medium comprising instructions that are executable by the processor to cause the processor to perform operations comprising:
receiving identity information about a first entity and contextual information about a second entity;
requesting a first set of data relating to the second entity from a first computing system using a diagnostic assistant tool;
transmitting a request for a second set of data relating to the second entity to a second computing system that is disjunctive with respect to the first computing system;
receiving the first set of data relating to the second entity from the first computing system and the second set of data from the second computing system;
de-duplicating the first set of data and the second set of data to generate de-duplicated data about the second entity; and
transmitting the de-duplicated data about the first entity to the diagnostic assistant tool to facilitate an interaction between the second entity and the first entity.

2. The system of claim 1, wherein the first entity includes a patient, and wherein the second entity includes a medical professional configured to provide medical care to the patient.

3. The system of claim 1, wherein the identity information about the first entity comprises personally identifiable information comprising at least one of a name of the first entity, a date of birth of the first entity, an address of the first entity, an email of the first entity, a telephone number of the first entity, a passport number of the first entity, a fingerprint of the first entity, a driver license number of the first entity, a credit card number of the first entity, a debit card number of the first entity, and a Social Security number of the first entity, and wherein the contextual information about the second entity includes at least one of a reason for the first entity visiting the second entity and a type of the second entity.

4. The system of claim 1, wherein the operations further comprise:
receiving first input including the identity information about the first entity and the contextual information about the second entity;
receiving second input from the second entity that indicates that the second entity selected the diagnostic assistant tool; and
initializing, in response to receiving the second input, the diagnostic assistant tool.

5. The system of claim 1, wherein the operations further comprise:
receiving first input including the identity information about the first entity and the contextual information about the second entity; and
automatically initializing, in response to receiving the first input, the diagnostic assistant tool.

6. The system of claim 1, wherein the first computing system includes an electronic medical records system, wherein the second computing system includes a provider database associated with a provider of the diagnostic assistant tool, and wherein the first set of data and the second set of data at least partially overlap.

7. The system of claim 1, wherein the operation of transmitting the de-duplicated data comprises:
generating, via the diagnostic assistant tool, a user interface; and
providing the de-duplicated data about the first entity via the user interface.

8. A method comprising:
receiving, by a computing device, identity information about a first entity and contextual information about a second entity;
requesting, by the computing device, a first set of data relating to the second entity from a first computing system using a diagnostic assistant tool;
transmitting, by the computing device, a request for a second set of data relating to the second entity to a second computing system that is disjunctive with respect to the first computing system;
receiving, by the computing device, the first set of data relating to the second entity from the first computing system and the second set of data from the second computing system;
de-duplicating, by the computing device, the first set of data and the second set of data to generate de-duplicated data about the second entity; and
transmitting, by the computing device, the de-duplicated data about the first entity to the diagnostic assistant tool to facilitate an interaction between the second entity and the first entity.

9. The method of claim 8, wherein the first entity includes a patient, and wherein the second entity includes a medical professional configured to provide medical care to the patient.

10. The method of claim 8, wherein the identity information about the first entity comprises personally identifiable information comprising at least one of a name of the first entity, a date of birth of the first entity, an address of the first entity, an email of the first entity, a telephone number of the first entity, a passport number of the first entity, a fingerprint of the first entity, a driver license number of the first entity, a credit card number of the first entity, a debit card number of the first entity, and a Social Security number of the first entity, and wherein the contextual information about the second entity includes at least one of a reason for the first entity visiting the second entity and a type of the second entity.

11. The method of claim 8, further comprising:
receiving, by the computing device, first input including the identity information about the first entity and the contextual information about the second entity;
receiving, by the computing device, second input from the second entity that indicates that the second entity selected the diagnostic assistant tool; and
initializing, by the computing device and in response to receiving the second input, the diagnostic assistant tool.

12. The method of claim 8, further comprising:
receiving, by the computing device, first input including the identity information about the first entity and the contextual information about the second entity; and
automatically initializing, by the computing device and in response to receiving the first input, the diagnostic assistant tool.

13. The method of claim 8, wherein the first computing system includes an electronic medical records system, wherein the second computing system includes a provider database associated with a provider of the diagnostic assistant tool, and wherein the first set of data and the second set of data at least partially overlap.

14. The method of claim 8, wherein transmitting the de-duplicated data comprises:
generating, by the computing device and via the diagnostic assistant tool, a user interface; and
providing, by the computing device, the de-duplicated data about the first entity via the user interface.

15. A non-transitory computer-readable medium comprising instructions that are executable by a processing device for causing the processing device to perform operations comprising:
receiving identity information about a first entity and contextual information about a second entity;
requesting a first set of data relating to the second entity from a first computing system using a diagnostic assistant tool;
transmitting a request for a second set of data relating to the second entity to a second computing system that is disjunctive with respect to the first computing system;
receiving the first set of data relating to the second entity from the first computing system and the second set of data from the second computing system;
de-duplicating the first set of data and the second set of data to generate de-duplicated data about the second entity; and
transmitting the de-duplicated data about the first entity to the diagnostic assistant tool to facilitate an interaction between the second entity and the first entity.

16. The non-transitory computer-readable medium of claim 15, wherein the first entity includes a patient, wherein the second entity includes a medical professional configured to provide medical care to the patient, wherein the identity information about the first entity comprises personally identifiable information comprising at least one of a name of the first entity, a date of birth of the first entity, an address of the first entity, an email of the first entity, a telephone number of the first entity, a passport number of the first entity, a fingerprint of the first entity, a driver license number of the first entity, a credit card number of the first entity, a debit card number of the first entity, and a Social Security number of the first entity, and wherein the contextual information about the second entity includes at least one of a reason for the first entity visiting the second entity and a type of the second entity.

17. The non-transitory computer-readable medium of claim 15, wherein the operations further comprise:
receiving first input including the identity information about the first entity and the contextual information about the second entity;
receiving second input from the second entity that indicates that the second entity selected the diagnostic assistant tool; and
initializing, in response to receiving the second input, the diagnostic assistant tool.

18. The non-transitory computer-readable medium of claim 15, wherein the operations further comprise:
receiving first input including the identity information about the first entity and the contextual information about the second entity; and
automatically initializing, in response to receiving the first input, the diagnostic assistant tool.

19. The non-transitory computer-readable medium of claim 15, wherein the first computing system includes an electronic medical records system, wherein the second computing system includes a provider database associated with a provider of the diagnostic assistant tool, and wherein the first set of data and the second set of data at least partially overlap.

20. The non-transitory computer-readable medium of claim 15, wherein the operation of transmitting the de-duplicated data comprises:
generating, by the diagnostic assistant tool, a user interface; and
providing the de-duplicated data about the first entity via the user interface.
